SEQUENCE LISTING (17 sequences)

SEQ ID NO: 1 - protein, 92 aa, Artificial Sequence
  Synthetic
KATGYTFSNYWVEWVKQRPGHGLEWIGEILPGISSTNYDEKFKDKATFTADTSSNTAYMQLSSLTSEDSAVYYCSRGRPAWSAFWGQGTTVT

SEQ ID NO: 2 - protein, 92 aa, Artificial Sequence
  Synthetic
TVTGYSITSDHAWSWIRQFPGNKLEWMGYISYSGSTSYNPSLKSRVSVTRDTSKNHFFLQLNSVTTEDTATYYCARAAFIGGWFAYWGQGTT

SEQ ID NO: 3 - protein, 92 aa, Artificial Sequence; identical to SEQ ID NO: 2
  Synthetic

SEQ ID NO: 4 - protein, 92 aa, Artificial Sequence
  Synthetic
AASGFAFSSYDMSWVRQTPEKRLEWVAYITSGGGSTYYPDTVKGRFTISRDNAKNTLYLQMSSLKSEDTAMYYCAGSRSWFAYWGQGTTVTV

SEQ ID NO: 5 - protein, 92 aa, Artificial Sequence
  Synthetic
TVTGYSITSDSAWNWIRQFPGNKLEWMGHISYSGSTSYNPSLKSRISITRDTSKDQFFLQLNSVTTEDTATYYCARGSYYGNFGFTYWGQGT

SEQ ID NO: 6 - protein, 92 aa, Artificial Sequence
  Synthetic
AASGFTFSYYGMSWVRQTPDKRLEWVATISSGGSYTYYPDSVKGRFTISRDNSENTLYLQMNSLKSEDTAMYFCARRGSSGDYYGMDYWGQG

SEQ ID NO: 7 - protein, 92 aa, Artificial Sequence
  Synthetic
KASGYTFTKYGMNWVKQAPGKGLQWMGWINTNTGEPTYAEEFKGRFAFSLETSASTAYLQINNLENADSATYFCTRGSSYFDYWGQGTTVTV

SEQ ID NO: 8 - protein, 92 aa, Artificial Sequence
  Synthetic
GDSVSLSCRASQSISNNLHWYQQKSHESPRLLIKYASQSISGIPSRFSGSGSGTDFTLSINSVETEDFGMYFCQQSNSWPLTFGGGTKLELK

SEQ ID NO: 9 - protein, 92 aa, Artificial Sequence
  Synthetic
GEKVTITCSASSSVSYMHWFQQKPGTSPKLWIYSTPNLASGVPARFSGSGSGTSYSLTISRMEAEDAATYYCQQRSSYPLTFGGGTKLELKQ

SEQ ID NO: 10 - protein, 92 aa, Artificial Sequence
  Synthetic
GQKVTITCSASSSVSYMHWYQQKSGTSPKPWIYEISKLASGVPARFSGSGSGTSYSLTISSMEAEDAAIYYCQQWNYPLITFGGGTKLELKR

SEQ ID NO: 11 - protein, 92 aa, Artificial Sequence
  Synthetic
GEGVSFSCRASQSIGTGIHWYQRRTNGSPRLLIKYASESISGIPSRFSGSGSGTDFTLTINSVESEDIADYYCQQSYSWPLTFGAGTKLELK

SEQ ID NO: 12 - protein, 91 aa, Artificial Sequence
  Synthetic
GETVTITCQASENIASDLAWYQKEGRSPQLLVYEARNLADGVPSRFSGSGSDTLYSLNIHSLQSEDVARYYCQHYYGTPWTFGGGTKPRAN

SEQ ID NO: 13 - protein, 92 aa, Artificial Sequence
  Synthetic
GEKVTMSCKSSQSLLYSNSQKNYLAWYQQKPGQSPKLLIYWASTRESGVPPRFTGSGSGTDFSLTISSVKAEDLAVYYCQQYYSFPRTFGGG

SEQ ID NO: 14 - protein, 92 aa, Artificial Sequence
  Synthetic
GQRATISYRASKSVSTSGYSYMHWNQQKPGQPPRLLIYLVSNLESGVPARFSGSGSGTDFTLNIHPVEEEDAATYYCQHIRELTRSVEAPSS

SEQ ID NO: 15 - protein, 440 aa, Artificial Sequence
  Synthetic
QVQLVQSGGGLVQPGGSLRLSCAASGFTVSSNYMSWVRQAPGKGLEWVSVIYSGGSTYYADSVKGRFTISRDNSKNTLYLQMNSLRAEDTAVYYCARGIAAAGLDYWGQGTLVTVSPASTKGPSVFPLAPSSKSTSGGTAALGCLVKDYFPEPVTVSWNSGALTSGVHTFPAVLQSSGLYSLSSVVTVPSSSLGTQTYVCNVNHKPSNTKVDKKVEPKSCDKTSELVLTQPPSVSAAPGQKVTISCSGSSSNIGNNYVYWYQQLPGTAPKLLIYRNNQRPSGVPDRFSGSKSGTSASLAISGLRSEDEADYYCAAWDDSLSGWVFGGGTKLTVLGQPKAAPSVTLFPPSSEELQANKATLVCLISDFYPGAVTVAWKADGSPVKAGVETTTPSKQSNNKYAASSYLSLTPEQWKSHRSYSCQVTHEGSTVEKTVAPTECS

SEQ ID NO: 16 - protein, 440 aa, Artificial Sequence
  Synthetic
QVQLVQSGGGLIQPGGSLRLSCAASGFTVSSNYMSWVRQAPGKGLEWVSVIYSGGSTYYADSVKGRFTISRDNSKNTLYLQMNSLRAEDTAVYYCARGIAAAGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTSELVLTQPPSVSGTPGQRVTISCSGSSSNIGSNYVYWYQQLPGTAPKLLIYRNNQRPSGVPDRFSGSKSGTSASLAISGLRSEDEADYYCAAWDDSLSGWVFGGGTKLTVLGQPKAAPSVTLFPPSSEELQANKATLVCLISDFYPGAVTVAWKADGSPVKAGVETTTPSKQSNNKYAASSYLSLTPEQWKSHRSYSCQVTHEGSTVEKTVAPTECS

SEQ ID NO: 17 - protein, 507 aa, Artificial Sequence
  Synthetic
MKKTAIAIAVALAGFATVAQAAELVLTQPPSVSGTPGQRVTISCSGSSSNIGSNYVYWYQQLPGTAPKLLIYRNNQRPSGVPDRFSGSKSGTSASLAISGLRSEDEADYYCAAWDDSLSGWVFGGGTKLTVLGQPKAAPSVTLFPPSSEELQANKATLVCLISDFYPGAVTVAWKADGSPVKAGVETTTPSKQSNNKYAASSYLSLTPEQWKSHRSYSCQVTHEGSTVEKTVAPTECSMKYLLPTAAAGLLLLAAQPAMAQVQLVQSGGGLIQPGGSLRLSCAASGFTVSSNYMSWVRQAPGKGLEWVSVIYSGGSTYYADSVKGRFTISRDNSKNTLYLQMNSLRAEDTAVYYCARGIAAAGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTSGQAGQHHHHHHGAYPYDVPDYAS

(19) United States
(12) Patent Application Publication
(10) Pub. No.: US 2012/0052007 A1
(43) Pub. Date: Mar. 1, 2012

(54) PERIPHERAL BLOOD SPARC BINDING ANTIBODIES AND USES THEREOF

(75) Inventors: Vuong Trieu, Agoura Hills, CA (US); Xiping Liu, Temple City, CA (US); Neil Desai, Los Angeles, CA (US)

(73) Assignee: ABRAXIS BIOSCIENCE, LLC, Los Angeles, CA (US)

(21) Appl. No.: 13/152,753

(22) Filed: Jun. 3, 2011

Related U.S. Application Data
(60) Provisional application No. 61/351,246, filed on Jun. 3, 2010.

(51) Int. Cl. A61K 51/10; A61K 9/127; A61P 19/00; A61K 39/395; C12N 9/96; A61P 13/12; A61P 37/02; A61P 35/00; A61P 35/02; A61P 9/00; A61P 13/00; A61K 9/14; C07K 16/18

(52) U.S. Cl. 424/1.49; 424/400; 424/450; 424/133.1; 424/174.1; 424/178.1; 424/183.1; 435/188; 530/389.7; 530/391.1; 530/391.3; 530/391.7

(57) ABSTRACT

The invention provides SPARC binding antibodies that have high affinity to SPARC, particularly plasma SPARC, and methods of using such antibodies in treating conditions including cancer.

### CROSS REFERENCE TO RELATED APPLICATIONS

This application claims priority benefit under 35 U.S.C. §119(e) of U.S. Provisional Application No. 61/351,246 filed on Jun. 3, 2010 the entire contents of which are incorporated herein by reference.

### BACKGROUND OF THE INVENTION

Secreted Protein, Acidic, Rich in Cysteines (SPARC), also known as osteonectin, is a 281 amino acid glycoprotein. SPARC has affinity for a wide variety of ligands including cations (e.g., Ca²⁺, Cu²⁺, Fe²⁺), growth factors (e.g., platelet derived growth factor (PDGF), and vascular endothelial growth factor (VEGF)), extracellular matrix (ECM) proteins (e.g., collagen I-V and collagen IX, vitronectin, and thrombospondin-1), endothelial cells, platelets, albumin, and hydroxyapaptite. SPARC expression is developmentally regulated, and is predominantly expressed in tissues undergoing remodeling during normal development or in response to injury (see, e.g., Lane et al., FASEB J., 8, 163-173 (1994)). High levels of SPARC protein are expressed in developing bones and teeth.

SPARC is a matricellular protein upregulated in several aggressive cancers, but is absent from the vast majority of normal tissues (Porter et al., J. Histochem. Cytochem., 43, 791 (1995) and see below). Indeed, SPARC expression is induced among a variety of tumors (e.g., bladder, liver, ovary, kidney, gut, and breast). In bladder cancer, for example, SPARC expression has been associated with advanced carcinoma. Invasive bladder tumors of stage T2 or greater have been shown to express higher levels of SPARC than bladder tumors of stage T1 (or less superficial tumors), and have poorer prognosis (see, e.g., Yamanaka et al., J. Urology, 166, 2495-2499 (2001)). In meningiomas, SPARC expression has been associated with invasive tumors only (see, e.g., Rempel et al., Clinical Cancer Res., 5, 237-241 (1999)). SPARC expression also has been detected in 74.5% of in situ invasive breast carcinoma lesions (see, e.g., Bellahcene, et al., Am. J. Pathol., 146, 95-100 (1995)), and 54.2% of infiltrating ductal carcinoma of the breast (see, e.g., Kim et al., J. Korean Med. Sci., 13, 652-657 (1998)). SPARC expression also has been associated with frequent microcalcification in breast cancer (see, e.g., Bellahcene et al., supra), suggesting that SPARC expression may be responsible for the affinity of breast metastases for the bone. SPARC is also known to bind albumin (see, e.g., Schnitzer, J. Biol. Chem., 269, 6072 (1994)).

Accordingly, there is a need for compositions and methods that take advantage of SPARC's role in disease and, in particular, SPARC's role in some cancers.

### BRIEF SUMMARY OF THE INVENTION

In one aspect, the invention provides compositions comprising a SPARC binding antibody, wherein the SPARC binding antibody comprises Imm12, Imm14, hHTI, or a combination thereof.

In another aspect, the invention provides methods of diagnosing or treating a disease, such as cancer, in an animal comprising administering a diagnostically or therapeutically effective amount of a composition comprising a SPARC binding antibody, wherein the SPARC binding antibody comprises Imm12, Imm14, hHTI, or a combination thereof.

The invention further provides methods of treating a tumor in an animal with one or more anticancer agents and a SPARC binding antibody comprising: isolating a biological sample from the animal, detecting the expression of SPARC protein in the biological sample, quantifying the amount of SPARC protein in the biological sample, if the SPARC protein in the biological sample is present above a threshold level administering a therapeutically effective amount of the anticancer agent and a therapeutically effective amount of the anti-SPARC antibody, or if the SPARC protein is present below the threshold level administering a therapeutically effective amount of the anticancer agent and none of the SPARC binding antibody. Suitable biological samples for quantifying the amount of SPARC expression for use in accordance with the invention include, e.g., blood, serum, and plasma. Suitable SPARC binding antibodies for treating a tumor in accordance with the invention include humanized SPARC binding antibodies, based on e.g., Imm12, Imm14, hHTI, and the like. Threshold levels for SPARC in the biological sample for the use of a SPARC binding antibody can be at least about 4.3 ng/ml, at least about 43 ng/ml, or, preferably, at least about 430 ng/ml.

In all methods and compositions of the present invention, the SPARC binding antibody can be conjugated to a therapeutic or diagnostic active agent. Suitable animals for administration of the compositions provided by the invention and application of the methods of the invention include, without limitation, human patients.

### BRIEF DESCRIPTION OF THE SEVERAL VIEWS OF THE DRAWING(S)

FIG. 1 is a restriction map of pASK84 used for cloning and expression of the Fab regions of Imm1 through Imm12.

FIG. 2 provides the amino acid sequences of two human SPARC binding Fab clones Fab6 and Fab16 (SEQ ID NOs 15-16).

FIG. 3 is a restriction map of the pBAD vector used for cloning and expression of Fab16.

FIG. 4 provides the amino acid sequences of Fab16 in pBad (SEQ ID NO: 17).

FIG. 5 is a restriction map of the pcDNA3002NEO vector used for the cloning and expression of fully-human antibodies Imm13 and Imm14 from Fab6 (SEQ ID NO: 15) and Fab16 (SEQ ID NO: 16).

FIG. 6 provides amino acid sequences of framework regions (FWRs) and complementarity determining regions (CDRs) for Imm1 (SEQ ID NOs 1 and 8), Imm2 (SEQ ID NOs 2 and 9), Imm3 (SEQ ID NOs 3 and 10), Imm4 (SEQ ID NOs 4 and 11), Imm6 (SEQ ID NO 5 and 12), Imm10 (SEQ ID NOs 6 and 13), and Imm12 (SEQ ID NOs 7 and 14).

FIG. 7 provides quantitative ELISA results of 1:1, 1:10, and 1:100 dilutions of Imm1 through Imm6 and Imm8 through Imm12 supernatants against human SPARC, as well as a control mAb.

FIG. 8 provides quantitative ELISA results of 0.04 μg/mL, 0.2 μg/mL, 1 μg/mL, and 5 μg/mL concentrations of purified Imm1 through Imm12 antibodies against human SPARC, as well as positive and negative controls.

FIG. 9 provides quantitative ELISA results comparing the binding of Imm1, Imm3, Imm4, Imm7, Imm9, and Imm10 antibodies to HTI-SPARC (platelet SPARC) and binding of Imm10, Imm11, Imm12, and control antibodies to Bio1-SPARC.

FIG. 10 provides quantitative ELISA results of Fab 16 binding to HTI-SPARC (platelet SPARC) and Bio1-SPARC at various concentrations.

FIG. 11 is a sensorgram prepared using surface plasmon resonance of Fab16 binding to human HTI SPARC.

FIG. 12 is a sensorgram prepared using surface plasmon resonance of Fab16 binding to human BIO1 SPARC.

FIG. 13 provides quantitative ELISA results of Imm11, Imm12, Imm13, and Imm14 binding against human SPARC at various concentrations.

FIG. 14 is a Western Blot of Imm series antibodies against denatured human SPARC.

FIG. 15 is a plot depicting the effect of SPARC binding antibodies mHTI (designated Imm17), Imm12, and Imm14, as well as control mIgG, on survival of animals bearing LL/2 Lewis Lung Carcinoma.

FIG. 16 provides quantitative ELISA results of Imm12, Imm14, and mHTI (designated Imm17) binding against human and murine SPARC.

FIG. 17A is a plot depicting overall survival of Cohort 1, patients having received prior chemotherapy (PC), for High SPARC and Low SPARC.

FIG. 17B is a plot depicting overall survival of Cohort 2, patients having received no prior chemotherapy (NPC), for High SPARC and Low SPARC.

FIG. 18 is a dot plot depicting SPARC levels in Cohort 1 (prior chemotherapy) and Cohort 2 (no prior chemotherapy), before and after treatment, as compared to normal controls.

FIG. 19 is a bar chart depicting percent change in plasma SPARC following treatment.

FIG. 20A is a plot depicting progression free survival (PFS) for patients in the High Risk cluster (Cluster 1) and Low Risk cluster (Cluster 2).

FIG. 20B is a plot depicting overall survival (OS) for patients in the High Risk cluster (Cluster 1) and Low Risk cluster (Cluster 2).

FIG. 21 is a dot plot depicting SPARC levels in Cohort 1 (prior chemotherapy) and Cohort 2 (no prior chemotherapy), for High Risk (HR) and Low Risk (LR) clusters.

FIG. 22A is a plot depicting progression free survival (PFS) for patients in Risk Levels 0, 1, of 2 as compared to all patients in the High Risk (HR) cluster.

FIG. 22B is a plot depicting overall survival (OS) for patients in Risk Levels 0, 1, of 2 as compared to all patients in the High Risk (HR) cluster.

FIG. 23A is a plot depicting tumor volume after treatment with 5-fluorouracil (5-FU) at 25 mg/kg and SPARC at 0.1 mg, 0.15 mg, or 0.20 mg, as compared to saline or 5-FU alone.

FIG. 23B is a plot depicting tumor volume after treatment with docetaxel (10 mg/kg) and SPARC at 0.2 mg, as compared to saline, docetaxel alone, or SPARC alone.

FIG. 24A is a plot depicting HT29 tumor volume after treatment with suntinib (SUT) (30 mg/kg), SPARC (BIO1) (0.2 mg), and nab-paclitaxel (ABX) (15 mg/kg) as compared to a negative control, SPARC alone, nab-paclitaxel alone, nab-paclitaxel and suntinib, and nab-paclitaxel and SPARC.

FIG. 24B is a plot depicting HT29 tumor volume after treatment with bevacizumab (AVS) (0.2 mg), SPARC (BIO1) (0.2 mg), and nab-paclitaxel (ABX) (15 mg/kg) as compared to a negative control, SPARC alone, nab-paclitaxel alone, nab-paclitaxel and bevacizumab, and nab-paclitaxel and SPARC.

FIG. 24C is a plot depicting MDA-MB-231 tumor volume after treatment with suntinib (SUT) (30 mg/kg), SPARC (BIO1) (0.2 mg), and nab-paclitaxel (ABX) (10 mg/kg) as compared to nab-paclitaxel alone, and nab-paclitaxel and suntinib.

FIG. 24D is a plot depicting PC3 tumor volume after treatment with bevacizumab (AVS) (0.2 mg), SPARC (BIO1) (0.2 mg), and nab-paclitaxel (ABX) (10 mg/kg) as compared to saline, SPARC alone, nab-paclitaxel alone, nab-paclitaxel and bevacizumab, and nab-paclitaxel and SPARC.

FIG. 25 is a bar graph depicting sprouts per bead upon administration of 0 μg/mL, 10 μg/mL, and 100 μg/mL of recombinant wild-type human SPARC (BIO1) as compared to negative control DPBS.

FIG. 26A depicts tubule formation at 0 μg/mL, 10 μg/mL, and 100 μg/mL of recombinant wild-type human SPARC (BIO1).

FIG. 26B is a dot plot depicting tube length at concentrations of 0 μg/mL, 10 μg/mL, and 100 μg/mL of recombinant wild-type human SPARC (BIO1) as compared to positive control VEGF and untreated tubules.

FIG. 27 is a dot plot depicting lung metastasis by protein levels in the MDA-MB-435-Luc+metastatic model for treatment with SPARC (4 mg/kg) and nab-paclitaxel (10 mg/kg), as compared to saline, SPARC alone, and nab-paclitaxel alone.

FIG. 28 provides photographs of tubule formation in LL2 metastasis tissue samples including SPARC (10 mg/mL) and Imm12, SPARC and Imm14, SPARC and mHTI (identified as Imm17), mIgG (negative control), no antibody (negative control), and in the absence of SPARC or antibody.

FIG. 29 provides quantitative fluorescence results for tumor localization of various antibodies including mHTI (designated “HTI”), Imm-12, and Imm-14, as compared to Imm-2 and Imm-3.

### DETAILED DESCRIPTION OF THE INVENTION

The present invention relates to certain SPARC binding antibodies which were analyzed for binding specificity to both human and murine SPARC, as well as ability to inhibit angiogenesis and metastasis. Surprisingly, the analysis revealed that although three of the antibodies bound native and denatured human SPARC in screening ELISA, only one antibody also bound murine SPARC. These three antibodies, Imm12, Imm14, and mHTI were also surprisingly found to have antiangiogenic (i.e., anti-tumor) and anti-metastatic properties.

### DEFINITIONS

“Peptide” and “polypeptide” are used interchangeably herein and refer to a compound made up of a chain of amino acid residues linked by peptide bonds. An “active portion” of a polypeptide means a peptide that is less than the full length polypeptide, but which retains measurable biological activity and retains biological detection.

As used herein, the term “tumor” refers to any neoplastic growth, proliferation or cell mass whether benign or malignant (cancerous), whether a primary site lesion or metastases.

As used herein, the term “cancer” refers to a proliferative disorder caused or characterized by a proliferation of cells which have lost susceptibility to normal growth control. Cancers of the same tissue type usually originate in the same tissue, and may be divided into different subtypes based on their biological characteristics. Four general categories of cancer are carcinoma (epithelial cell derived), sarcoma (connective tissue or mesodermal derived), leukemia (blood-forming tissue derived) and lymphoma (lymph tissue derived). Over 200 different types of cancers are known, and every organ and tissue of the body may be affected. Specific examples of cancers that do not limit the definition of cancer may include melanoma, leukemia, astrocytoma, glioblastoma, retinoblastoma, lymphoma, glioma, Hodgkin's lymphoma, and chronic lymphocytic leukemia. Examples of organs and tissues that may be affected by various cancers include pancreas, breast, thyroid, ovary, uterus, testis, prostate, pituitary gland, adrenal gland, kidney, stomach, esophagus, rectum, small intestine, colon, liver, gall bladder, head and neck, tongue, mouth, eye and orbit, bone, joints, brain, nervous system, skin, blood, nasopharyngeal tissue, lung, larynx, urinary tract, cervix, vagina, exocrine glands, and endocrine glands. Alternatively, a cancer can be multicentric or of unknown primary site (CUPS).

As used herein “a suitable SPARC binding antibody” or “a SPARC binding antibody” refers to an antibody capable of binding to SPARC with specificity.

As used herein “tumor targeting antibody” refers to a disease targeting antibody wherein the disease is a tumor, cancer, neoplasm or the like.

As used here, “SPARC binding antibody” refers to antibody that has affinity for circulating SPARC with Kd in the range of 1, or 10, or 100, or 1000 nM—preferably less than or equal to 10 nM.

As used herein “therapeutically effective amount” refers to an amount of a composition that relieves (to some extent, as judged by a skilled medical practitioner) one or more symptoms of the disease or condition in a mammal. Additionally, by “therapeutically effective amount” of a composition is meant an amount that returns to normal, either partially or completely, physiological or biochemical parameters associated with or causative of a disease or condition. A clinician skilled in the art can determine the therapeutically effective amount of a composition in order to treat or prevent a particular disease condition, or disorder when it is administered, such as intravenously, subcutaneously, intraperitoneally, orally, or through inhalation. The precise amount of the composition required to be therapeutically effective will depend upon numerous factors, e.g., such as the specific activity of the active agent, the delivery device employed, physical characteristics of the agent, purpose for the administration, in addition to many patient specific considerations. But a determination of a therapeutically effective amount is within the skill of an ordinarily skilled clinician upon the appreciation of the disclosure set forth herein.

The terms “treating,” “treatment,” “therapy,” and “therapeutic treatment” as used herein refer to curative therapy, prophylactic therapy, or preventative therapy. An example of “preventative therapy” is the prevention or lessening the chance of a targeted disease (e.g., cancer or other proliferative disease) or related condition thereto. Those in need of treatment include those already with the disease or condition as well as those prone to have the disease or condition to be prevented. The terms “treating,” “treatment,” “therapy,” and “therapeutic treatment” as used herein also describe the management and care of a mammal for the purpose of combating a disease, or related condition, and includes the administration of a composition to alleviate the symptoms, side effects, or other complications of the disease, condition. Therapeutic treatment for cancer includes, but is not limited to, surgery, chemotherapy, radiation therapy, gene therapy, and immunotherapy.

As used herein, the term “agent” or “drug” or “therapeutic agent” refers to a chemical compound, a mixture of chemical compounds, a biological macromolecule, or an extract made from biological materials such as bacteria, plants, fungi, or animal (particularly mammalian) cells or tissues that are suspected of having therapeutic properties. The agent or drug can be purified, substantially purified or partially purified. An “agent” according to the present invention, also includes a radiation therapy agent or a “chemotherapuetic agent.”

As used herein, the term “diagnostic agent” refers to agents allowing for the quantitation of plasma/circulating SPARC by methods such as ELISA.

As used herein, the term “chemotherapuetic agent” refers to an agent with activity against cancer, neoplastic, and/or proliferative diseases.

As used herein, the term “radiotherapeutic regimen” or “radiotherapy” refers to the administration of radiation to kill cancerous cells. Radiation interacts with various molecules within the cell, but the primary target, which results in cell death is the deoxyribonucleic acid (DNA). However, radiotherapy often also results in damage to the cellular and nuclear membranes and other organelles. DNA damage usually involves single and double strand breaks in the sugar-phosphate backbone. Furthermore, there can be cross-linking of DNA and proteins, which can disrupt cell function. Depending on the radiation type, the mechanism of DNA damage may vary as does the relative biologic effectiveness. For example, heavy particles (i.e. protons, neutrons) damage DNA directly and have a greater relative biologic effectiveness. Whereas, electromagnetic radiation results in indirect ionization acting through short-lived, hydroxyl free radicals produced primarily by the ionization of cellular water. Clinical applications of radiation consist of external beam radiation (from an outside source) and brachytherapy (using a source of radiation implanted or inserted into the patient). External beam radiation consists of X-rays and/or gamma rays, while brachytherapy employs radioactive nuclei that decay and emit alpha particles, or beta particles along with a gamma ray.

As used herein the term “alternative therapeutic regimen” or “alternative therapy” (not a first line chemotherapeutic regimen as described above) may include for example, receptor tyrosine kinase inhibitors (for example Iressa™ (gefitinib), Tarceva™ (erlotinib), Erbitux™ (cetuximab), imatinib mesilate (Gleevec™)); proteosome inhibitors (for example bortezomib (Velcade™)); VEGFR2 inhibitors such as PTK787 (ZK222584), aurora kinase inhibitors (for example ZM447439); mammalian target of rapamycin (mTOR) inhibitors, cyclooxygenase-2 (COX-2) inhibitors, rapamycin inhibitors (for example sirolimus, (Rapamune™)); farnesyltransferase inhibitors (for example tipifarnib (Zarnestra™)); matrix metalloproteinase inhibitors (for example BAY 12-9566; sulfated polysaccharide tecogalan); angiogenesis inhibitors (for example Avastin™ (bevacizumab); analogues of fumagillin such as TNP-4; carboxyaminotriazole; BB-94 and BB-2516; thalidomide; interleukin-12; linomide; peptide fragments; and antibodies to vascular growth factors and vascular growth factor receptors); platelet derived growth factor receptor inhibitors, protein kinase C inhibitors, mitogen-activated kinase inhibitors, mitogen-activated protein kinase kinase inhibitors, Rouse sarcoma virus transforming oncogene (SRC) inhibitors, histonedeacetylase inhibitors, small hypoxia-inducible factor inhibitors, hedgehog inhibitors, and TGF-β signaling inhibitors. Furthermore, an immunotherapeutic agent would also be considered an alternative therapeutic regimen. For example, serum or gamma globulin containing preformed antibodies; nonspecific immunostimulating adjuvants; active specific immunotherapy; and adoptive immunotherapy. In addition, alternative therapies may include other biological-based chemical entities such as polynucleotides, including antisense molecules, polypeptides, antibodies, gene therapy vectors and the like. Such alternative therapeutics may be administered alone or in combination, or in combination with other therapeutic regimens described herein. Methods of use of chemotherapeutic agents and other agents used in alternative therapeutic regimens in combination therapies, including dosing and administration regimens, will also be known to a one skilled in the art.

As used herein the term “tumor localization” means the degree to which, upon injection into a tumor bearing animal, a SPARC binding antibody concentrates in a SPARC-expressing tumor. Tumor localization may be measured by any suitable method including, but not limited to, labeling the antibody with a fluorescent dye, injecting the now fluorescent antibody into an animal with a tumor and determining a ratio of tumor fluorescence to the fluorescence from skin away from any gross tumor, wherein localization is present if said ratio is >20, preferably >10, more preferably >5.

Antibodies

The invention provides a SPARC binding antibody. In particular, the SPARC binding antibody can be Imm12, Imm14, mHTI, hHTI (a humanized version of mHTI), or combinations thereof.

In addition, the invention provides for a SPARC binding antibody capable of binding both SPARC found in the blood, e.g. HTI (platelet) SPARC and SPARC found at a tumor site, e.g. Bio1-SPARC. Various methods of determining antibody binding strength are known to those of ordinary skill in the art.

For human use, in order to avoid immunogenicity and immune response, it is preferable to use a humanized SPARC binding antibody or suitable fragments such as Fab′, Fab, or Fab2. Humanized antibody or fragments thereof can be produced, for example, using one of the following established methods: 1) a humanized antibody can be constructed using human IgG backbone replacing the variable CDR region with that of an antibody against SPARC, where the heavy and light chain are independently expressed under separate promoters or coexpressed under one promoter with an IRES sequence; 2) a humanized monoclonal antibody can be raised against SPARC using a mouse engineered to have a human immune system; 3) a humanized antibody against SPARC can be raised using phagemid (M13, lambda coliphage, or any phage system capable of surface presentation). To construct the full length antibody, the variable region can be transferred onto the CDR of both a heavy chain and a light chain. The coexpression of the heavy chain and light chain in mammalian cells such as CHO, 293, or human myeloid cells can provide a full length antibody. Similarly, Fab′, Fab, or Fab2 fragments and single chain antibodies can be prepared using well established methods.

The present invention further provides a humanized antibody that specifically recognizes the epitopes unique to plasma SPARC. The humanized antibody is typically a human antibody in which residues from CDRs are replaced with residues from CDRs of a non-human species such as mouse, rat or rabbit having the desired specificity, affinity and capacity. In some instances, Fv framework residues of the human antibody are replaced by corresponding non-human residues. Any suitable monoclonal antibody can be used as a source of CDRs, for example, an antiSPARC antibody that binds to both human and murine SPARC and, in particular, binds circulating human SPARC, shows good antiangiogenic activity in vitro assays, and has promising results in animal models (e.g., reduces metastases in xenograft model systems.)

There are four general steps to humanize a monoclonal antibody. These are: (1) determining the nucleotide and predicted amino acid sequence of the starting antibody light and heavy variable domains (2) designing the humanized antibody, i.e., deciding which antibody framework region to use during the humanizing process (3) the actual humanizing methodologies/techniques and (4) the transfection and expression of the humanized antibody. See, for example, U.S. Pat. Nos. 4,816,567; 5,807,715; 5,866,692; 6,331,415; 5,530,101; 5,693,761; 5,693,762; 5,585,089; 6,180,370; and 6,548,640 (which are hereby incorporated by reference.) For example, the constant region may be engineered to more resemble human constant regions to avoid immune response if the antibody is used in clinical trials and treatments in humans. See, for example, U.S. Pat. Nos. 5,997,867 and 5,866,692 (which are hereby incorporated by reference.)

It is important that antibodies be humanized with retention of high affinity for the antigen and other favorable biological properties. To achieve this goal, humanized antibodies can be prepared by a process of analysis of the parental sequences and various conceptual humanized products using three dimensional models of the parental and humanized sequences. Three dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e. the analysis of residues that influence the ability of the candidate immunoglobulin to bind its antigen. In this way, framework residues can be selected and combined from the consensus and import sequence so that the desired antibody characteristic, such as increased affinity for the target antigen(s), is achieved. In general, the CDR residues are directly and most substantially involved in influencing antigen binding. The humanized antibodies may also contain modifications in the hinge region to improve one or more characteristics of the antibody.

Alternatively, antibodies may be screened and made recombinantly by phage display technology. See, for example, U.S. Pat. Nos. 5,565,332; 5,580,717; 5,733,743 and 6,265,150 (which are hereby incorporated by reference.) Alternatively, the phage display technology (McCafferty et al., Nature 348:552-553 (1990)) can be used to produce human antibodies and antibody fragments in vitro, from immunoglobulin variable (V) domain gene repertoires from unimmunized donors.

In a natural immune response, antibody genes accumulate mutations at a high rate (somatic hypermutation). Some of the changes introduced will confer higher affinity, and B cells displaying high-affinity surface immunoglobulin are preferentially replicated and differentiated during subsequent antigen challenge. This natural process can be mimicked by employing the technique known as “chain shuffling.” Marks, et al., Bio/Technol. 10:779-783 (1992)). In this method, the affinity of “primary” human antibodies obtained by phage display can be improved by sequentially replacing the heavy and light chain V region genes with repertoires of naturally occurring variants (repertoires) of V domain genes obtained from unimmunized donors. This technique allows the production of antibodies and antibody fragments with affinities in the pM-nM range.

Gene shuffling can also be used to derive human antibodies from rodent antibodies, where the human antibody has similar affinities and specificities to the starting rodent antibody. According to this method, which is also referred to as “epitope imprinting,” the heavy or light chain V domain gene of rodent antibodies obtained by phage display technique is replaced with a repertoire of human V domain genes, creating rodent-human chimeras. Selection on antigen results in isolation of human variable regions capable of restoring a functional antigen-binding site, i.e., the epitope governs (imprints) the choice of partner. When the process is repeated in order to replace the remaining rodent V domain, a human antibody is obtained (see PCT Publication No. WO 93/06213, published Apr. 1, 1993). Unlike traditional humanization of rodent antibodies by CDR grafting, this technique provides completely human antibodies, which have no framework or CDR residues of rodent origin. It is apparent that although the above discussion pertains to humanized antibodies, the general principles discussed are applicable to customizing antibodies for use, for example, in dogs, cats, primates, equines and bovines.

The SPARC binding antibodies of the present invention include whole antibodies as well as fragments of the antibody retaining the binding site for SPARC (e.g., Fab′, Fab and Fab2). The antibody can be any class of antibody, e.g., IgM, IgA, IgG, IgE, IgD, and IgY. The antibody can be, for example, a divalent, monovalent, or chimeric antibody with one valence for SPARC and another for an active agent (such as tTF or ricin A, or another active agent as described herein). The humanized antibody is not limited to IgG. The same technologies can be used to generate all other classes of antibodies such as IgE, IgA, IgD, IgM, each having different antibody-dependent cellular cytotoxicity (ADCC) and complement dependent cytotoxicity (CDC) activities appropriate to particular disease target. Functional fragments of the antibody can be generated by limited proteolysis. These fragments can be monovalent such as Fab′ or divalent, such as Fab2. Fragments can also be synthesized as single chain scfv or diabodies in E. coli.

Compositions

The invention provides a composition comprising a SPARC binding antibody as described above. In some embodiments, the composition comprises Imm12, Imm14, mHTI, or hHTI, along with a suitable carrier. In other embodiments, the composition comprises a combination of Imm12, Imm14, mHTI, or hHTI, along with a suitable carrier. In preferred embodiments, the composition is a pharmaceutically acceptable composition comprising a SPARC binding antibody and a pharmaceutically acceptable carrier.

The compositions of the present invention can further comprise an active agent. In some embodiments, the active agent is a pharmaceutically active therapeutic agent directly able to exert its pharmacological effect. In other embodiments, the active agent is a diagnostic agent. In preferred embodiments, the active agent is a diagnostic or therapeutic active agent conjugated or administered together with SPARC binding antibody. It will be understood that some active agents are useful as both diagnostic and therapeutic agents, and therefore such terms are not mutually exclusive.

Compositions of the present invention can be used to enhance delivery of the active agent to a disease site relative to delivery of the active agent alone, or to enhance SPARC clearance resulting in a decrease in blood level of SPARC. In preferred embodiments, the decrease in blood level of SPARC is at least about 10%. In more preferred embodiments, the decrease in blood level of SPARC is at least about 15%, 20%, 25%, 30%, 35%, 40%, 45%, or, most preferably, at least about 50%.

The active agent can be any suitable therapeutic agent or diagnostic agent, such as a chemotherapeutic or anticancer agent. Suitable diagnostic agents include fluorochromes, radioactive agents, MRI contrast agents, X-ray contrast agents, ultrasound contrast agents, and PET contrast agents. Suitable chemotherapeutic agents or other anticancer agents for use in accordance with the invention include, but are not limited to, tyrosine kinase inhibitors (genistein), biologically active agents (TNF, tTF), radionuclides (1311, 90Y, 111In, 211At, 32P and other known therapeutic radionuclides), adriamycin, ansamycin antibiotics, asparaginase, bleomycin, busulphan, cisplatin, carboplatin, carmustine, capecitabine, chlorambucil, cytarabine, cyclophosphamide, camptothecin, dacarbazine, dactinomycin, daunorubicin, dexrazoxane, docetaxel, doxorubicin, etoposide, epothilones, floxuridine, fludarabine, fluorouracil, gemcitabine, hydroxyurea, idarubicin, ifosfamide, irinotecan, lomustine, mechlorethamine, mercaptopurine, meplhalan, methotrexate, rapamycin (sirolimus) and derivatives, mitomycin, mitotane, mitoxantrone, nitrosurea, paclitaxel, pamidronate, pentostatin, plicamycin, procarbazine, rituximab, streptozocin, teniposide, thioguanine, thiotepa, taxanes, vinblastine, vincristine, vinorelbine, taxol, combretastatins, discodemiolides, and transplatinum.

Other suitable chemotherapeutic agents for use in accordance with invention include, without limitation, antimetabolites (e.g., asparaginase), antimitotics (e.g., vinca alkaloids), DNA damaging agents (e.g., cisplatin), proapoptotics (agents which induce programmed-cell-death or apoptosis) (e.g, epipodophylotoxins), differentiation inducing agents (e.g., retinoids), antibiotics (e.g., bleomycin), and hormones (e.g., tamoxifen, diethylstibestrol). Further, suitable chemotherapeutic agents for use in accordance with the invention include antiangiogenesis agents (angiogenesis inhibitors) such as, e.g., IFN-alpha, fumagillin, angiostatin, endostatin, thalidomide, and the like. Many other anti-angiogenic agents have been identified and are known in the art, including those listed by Carmeliet and Jain (2000). The anti-angiogenic agent can be naturally occurring or non-naturally occurring. In some embodiments, the chemotherapeutic agent is a synthetic antiangiogenic peptide. For example, it has been previously reported that the antiangiogenic activity of small synthetic pro-apoptotic peptides comprises two functional domains, one targeting the CD13 receptors (aminopeptidase N) on tumor microvessels and the other disrupting the mitochondrial membrane following internalization. Nat. Med. 1999, 5(9):1032-8. In other embodiments, the anti-angiogenic agent is a second generation dimeric peptide, CNGRC-GG-d(KLAKLAK)₂, named HKP (Hunter Killer Peptide) was found to have improved antitumor activity. In certain embodiments, the antiangiogenic agent is other than an anti-VEGF antibody (such as bevacizumab) although one of ordinary skill in the art will also understand that bevacizumab could be used in accordance with the invention.

Preferred chemotherapeutic agents include docetaxel, paclitaxel, and combinations thereof. “Combinations thereof” refers to both the administration of dosage forms including more than one drug, for example, docetaxel and paclitaxel, as well as the sequential but, temporally distinct, administration of docetaxel and paclitaxel (e.g., the use of docetaxel in one cycle and paclitaxel in the next). Particularly preferred chemotherapeutic agents comprise particles of protein-bound drug, including but not limited to, wherein the protein making up the protein-bound drug particles comprises albumin including wherein more than 50% of the chemotherapeutic agent is in nanoparticle form. Most preferably the chemotherapeutic agent comprises particles of albumin-bound paclitaxel, such as, e.g., Abraxane®. Such albumin-bound paclitaxel formulations, denoted “nab-paclitaxel,” can be used in accordance with the invention where the paclitaxel dose administered is from about 30 mg/m² to about 1000 mg/m² with a dosing cycle of about 3 weeks (i.e., administration of the paclitaxel dose once every about three weeks). Further, it is desirable that the paclitaxel dose administered is from about 50 mg/m² to about 800 mg/m², preferably from about 80 mg/m2 to about 700 mg/m², and most preferably from about 250 mg/m² to about 300 mg/m² with a dosing cycle of about 3 weeks.

Other therapeutic agents also include, without limitation, biologically active polypeptides, antibodies and fragments thereof, lectins, and toxins (such as ricin A), or radionuclides. Suitable antibodies for use as active agents in accordance with the invention include, without limitation, conjugated (coupled) or unconjugated (uncoupled) antibodies, monoclonal or polyclonal antibodies, humanized or unhumanized antibodies, as well as Fab′, Fab, or Fab2 fragments, single chain antibodies and the like. Contemplated antibodies or antibody fragments can be Fc fragments of IgG, IgA, IgD, IgE, or IgM. In various preferred embodiments, the active agent is the Fc fragment of the antibody itself, a single chain antibody, a Fab fragment, diabody, and the like. In more preferred embodiments, the antibody or antibody fragment mediates complement activation, cell mediated cytotoxicity, opsonization, mast cell activation, and/or other immune response.

In addition, the pharmaceutically active agent can be an siRNA. In preferred embodiments, the siRNA molecule inhibits expression of an gene associated with tumors such as, for example, c-Sis and other growth factors, EGFR, PDGFR, VEGFR, HER2, other receptor tyrosine kinases, Src-family genes, Syk-ZAP-70 family genes, BTK family genes, other cytoplasmic tyrosine kinases, Raf kinase, cyclin dependent kinases, other cytoplasmic serine/threonine kinases, Ras protein and other regulatory GTPases.

SPARC binding antibodies can also be conjugated to polyethylene glycol (PEG). PEG conjugation can increase the circulating half-life of a protein, reduce the protein's immunogenicity and antigenicity, and improve the bioactivity. Any suitable method of conjugation can be used, including but not limited to, e.g., reacting methoxy-PEG with a SPARC binding antibody's available amino groups or other reactive sites such as, e.g., histidines or cysteines. In addition, recombinant DNA approaches can be used to add amino acids with PEG-reactive groups to the inventive SPARC binding antibodies. PEG can be processed prior to reacting it with a SPARC binding antibody, e.g., linker groups can be added to the PEG. Further, releasable and hybrid PEG-ylation strategies can be used in accordance with the invention, such as, e.g., the PEG-ylation of a SPARC binding antibody such that the PEG molecules added to certain sites in the SPARC binding antibody are released in vivo. Such PEG conjugation methods are known in the art (See, e.g., Greenwald et al., Adv. Drug Delivery Rev. 55:217-250 (2003)).

Contemplated SPARC binding antibodies and conjugates thereof can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such as organic acids as acetic, oxalic, tartaric, mandelic, and the like. Salts formed with the free carboxyl groups also can be derived from inorganic bases such as, for example, sodium, potassium, ammonium, calcium, or ferric hydroxides, and such organic bases as isopropylamine, trimethylamine, histidine, procaine and the like.

The compositions of the present inventions are generally provided in a formulation with a carrier, such as a pharmaceutically acceptable carrier. Typically, the carrier will be liquid, but also can be solid, or a combination of liquid and solid components. The carrier desirably is a physiologically acceptable (e.g., a pharmaceutically or pharmacologically acceptable) carrier (e.g., excipient or diluent). Suitable pharmaceutical excipients include stabilizers, antioxidants, osmolality adjusting agents, buffers, and pH adjusting agents. Suitable additives include physiologically biocompatible buffers, additions of chelants or calcium chelate complexes, or, optionally, additions of calcium or sodium salts. Pharmaceutical compositions can be packaged for use in liquid form, or can be lyophilized. Preferred physiologically acceptable carrier media are water, buffered water, normal saline, 0.4% saline, 0.3% glycine, hyaluronic acid and the like. Physiologically acceptable carriers are well known and are readily available. The choice of carrier will be determined, at least in part, by the location of the target tissue and/or cells, and the particular method used to administer the composition.

The composition can be formulated for administration by a route including intravenous, intraarterial, intramuscular, intraperitoneal, intrathecal, epidural, topical, percutaneous, subcutaneous, transmucosal (including, for example, pulmonary), intranasal, rectal, vaginal, or oral. The composition also can comprise additional components such as diluents, adjuvants, excipients, preservatives, and pH adjusting agents, and the like.

Formulations suitable for injectable administration include aqueous and nonaqueous, isotonic sterile injection solutions, which can contain anti-oxidants, buffers, bacteriostats, and solutes that render the formulation isotonic with the blood of the intended recipient, and aqueous and nonaqueous sterile suspensions that can include suspending agents, solubilizers, thickening agents, stabilizers, lyoprotectants, and preservatives. The formulations can be presented in unit-dose or multi-dose sealed containers, such as ampules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, or tablets.

Sterile injectable solutions can be prepared by incorporating the active compound in the required amount in an appropriate solvent with one or a combination of ingredients enumerated above, as required, followed by filtered sterilization. Preferably solutions for injection are free of endotoxin. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof. In all cases, the formulation must be sterile and must be fluid to the extent that easy syringability exists. It must be stable under the conditions of manufacture and storage and must be preserved against the contaminating action of microorganisms, such as bacteria and fungi. Solutions of the active compounds as free base or pharmacologically acceptable salts can be prepared in water suitably mixed with a surfactant, such as hydroxycellulose. Dispersions can also be prepared in glycerol, liquid polyethylene glycols, and mixtures thereof and in oils. Under ordinary conditions of storage and use, these preparations contain a preservative to prevent the growth of microorganisms.

In preferred embodiments, the active ingredients can be entrapped in microcapsules prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980). Specifically, liposomes containing the SPARC binding antibodies can be prepared by such methods as described in Rezler et al., J. Am. Chem. Soc. 129(16): 4961-72 (2007); Samad et al., Curr. Drug Deliv. 4(4): 297-305 (2007); and U.S. Pat. Nos. 4,485,045 and 4,544,545. Liposomes with enhanced circulation time are disclosed in U.S. Pat. No. 5,013,556.

Particularly useful liposomes can be generated by, for example, the reverse-phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Polypeptides of the present invention can be conjugated to the liposomes as described in Werle et al., Int. J. Pharm. 370(1-2): 26-32 (2009).

In other embodiments, a composition can be delivered using a natural virus or virus-like particle, a dendrimer, carbon nanoassembly, a polymer carrier, a paramagnetic particle, a ferromagnetic particle, a polymersome, a filomicelle, a micelle or a lipoprotein.

Administration into the airways can provide either systemic or local administration, for example to the trachea and/or the lungs. Such administration can be made via inhalation or via physical application, using aerosols, solutions, and devices such as a bronchoscope. For inhalation, the compositions herein are conveniently delivered from an insufflator, a nebulizer, a pump, a pressurized pack, or other convenient means of delivering an aerosol, non-aerosol spray of a powder, or non-aerosol spray of a liquid. Pressurized packs can comprise a suitable propellant such a liquefied gas or a compressed gas. Liquefied gases include, for example, fluorinated chlorinated hydrocarbons, hydrochlorofluorocarbons, hydrochlorocarbons, hydrocarbons, and hydrocarbon ethers. Compressed gases include, for example, nitrogen, nitrous oxide, and carbon dioxide. In particular, the use of dichlorodifluoromethane, trichlorofluoromethane, dichlorotetrafluoroethane, carbon dioxide or other suitable gas is contemplated. In the case of a pressurized aerosol, the dosage unit can be determined by providing a valve to deliver a controlled amount. In administering a dry powder composition, the powder mix can include a suitable powder base such as lactose or starch. The powder composition can be presented in unit dosage form such as, for example, capsules, cartridges, or blister packs from which the powder can be administered with the aid of an inhalator or insufflator.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays, inhaled aerosols, rectal or vaginal suppositories, mouthwashes, rapidly dissolving tablets, or lozenges. For transdermal administration, the active compounds are formulated into ointments, salves, gels, foams, or creams as generally known in the art.

The pharmaceutical compositions can be delivered using drug delivery systems. Such delivery systems include hyaluronic acid solutions or suspensions of collagen fragments. The drugs can be formulated in microcapsules, designed with appropriate polymeric materials for controlled release, such as polylactic acid, ethylhydroxycellulose, polycaprolactone, polycaprolactone diol, polylysine, polyglycolic, polymaleic acid, poly[N-(2-hydroxypropyl)methylacrylamide] and the like. Particular formulations using drug delivery systems can be in the form of liquid suspensions, ointments, complexes to a bandage, collagen shield or the like.

The composition can further comprise any other suitable components, especially for enhancing the stability of the composition and/or its end-use. Accordingly, there is a wide variety of suitable formulations of the composition of the invention.

Sustained release compositions can also be employed in the present compositions, such as those described in, for example, U.S. Pat. Nos. 5,672,659 and 5,595,760. The use of immediate or sustained release compositions depends on the nature of the condition being treated. If the condition consists of an acute or over-acute disorder, treatment with an immediate release form will be preferred over a prolonged release composition. Alternatively, for certain preventative or long-term treatments, a sustained release composition may be appropriate.

In addition, the composition can comprise additional therapeutic or biologically-active agents. For example, therapeutic factors useful in the treatment of a particular indication can be present. Factors that control inflammation, such as ibuprofen or steroids, can be part of the composition to reduce swelling and inflammation associated with in vivo administration of the pharmaceutical composition and physiological distress.

Compositions provided by the invention can include, e.g., from about 0.5 mL to about 4 mL aqueous or organic liquids with an active agent coupled to a SPARC binding antibody, with the concentration of the active agent from about 10 mg/mL to about 100 mg/mL, preferably from about 1 mg/mL to about 10 mg/mL, more preferably from about 0.1 mg/mL to about 1 mg/mL. The active agent can be present at any suitable and therapeutically effective concentration, e.g., bevacizumab at a concentration of from about 10 mg/mL to about 50 mg/mL.

Methods

The invention provides a method for diagnosing or treating a disease in an animal by administering a diagnostically or therapeutically effective amount of a composition comprising a SPARC binding antibody comprising Imm12, Imm14, mHTI, hHTI, or combinations thereof. In some embodiments, the invention provides a method for diagnosing a disease in an animal by administering an effective amount of Imm12, Imm14, mHTI, hHTI, or a combination thereof. In other embodiments, the invention provides a method for treating a disease in an animal by administering an effective amount of Imm12, Imm14, mHTI, hHTI, or a combination thereof. Any composition described above can be used in the methods of the present invention.

According to the methods of the present invention, a therapeutically effective amount of the composition can be administered to the mammal to enhance delivery of the active agent to a disease site relative to delivery of the active agent alone, or to enhance clearance resulting in a decrease in blood level of SPARC. In preferred embodiments, the decrease in blood level of SPARC is at least about 10%. In more preferred embodiments, the decrease in blood level of SPARC is at least about 15%, 20%, 25%, 30%, 35%, 40%, 45%, or, most preferably, at least about 50%.

The invention also provides a method of diagnosing a disease or condition in an animal comprising (a) administering to the animal a diagnostically effective amount of a SPARC binding antibody comprising Imm12, Imm14, mHTI, hHTI, or a combination thereof; (b) detecting the amount of SPARC binding antibody present in a particular site or tissue of the animal; and (c) diagnosing that the disease or condition is present if the amount of SPARC binding antibody present indicates that significantly greater than normal levels of SPARC are present in the particular site or tissue.

Likewise, the invention further provides methods of treating a tumor in an animal with one or more anticancer agents and a SPARC binding antibody comprising: isolating a biological sample from the animal, detecting the expression of SPARC protein in the biological sample, quantifying the amount of SPARC protein in the biological sample, if the SPARC protein in the biological sample is present above a threshold level administering a therapeutically effective amount of the anticancer agent and a therapeutically effective amount of the anti-SPARC antibody, or if the SPARC protein is present below the threshold level administering a therapeutically effective amount of the anticancer agent and none of the SPARC binding antibody.

The level of SPARC protein present in a sample is typically detected using an anti-SPARC antibody. However, in some embodiments, the expression of a SPARC protein can be determined using only a portion of an antibody, using a SPARC binding molecule which is not an antibody, or using some other method of detecting SPARC expression not requiring an antibody or a SPARC binding molecule.

The present methods can be used in any condition characterized by overexpression of SPARC. Exemplary diseases for which the present invention is useful include abnormal conditions of proliferation, tissue remodeling, hyperplasia, exaggerated wound healing in any bodily tissue including soft tissue, connective tissue, bone, solid organs, blood vessel and the like. Examples of diseases treatable or diagnosed using the methods and compositions of the present invention include cancer, diabetic or other retinopathy, inflammation, arthritis, restenosis in blood vessels or artificial blood vessel grafts or intravascular devices and the like.

Other diseases within the scope of the methods of the present invention include, without limitation, cancer, restenosis or other proliferative diseases, fibrosis, osteoporosis or exaggerated wound healing. Specifically, such suitable diseases include, without limitation, wherein: (a) the cancer can be, for example, carcinoma in situ, atypical hyperplasia, carcinoma, sarcoma, carcinosarcoma, lung cancer, pancreatic cancer, skin cancer, hematological neoplasms, breast cancer, brain cancer, colon cancer, bladder cancer, cervical cancer, endometrial cancer, esophageal cancer, gastric cancer, head and neck cancer, multiple myeloma, liver cancer, leukemia, lymphoma, oral cancer, osteosarcomas, ovarian cancer, prostate cancer, testicular cancer, and thyroid cancer, (b) the restenosis can be, for example, coronary artery restenosis, cerebral artery restenosis, carotid artery restenosis, renal artery restenosis, femoral artery restenosis, peripheral artery restenosis or combinations thereof, (c) the other proliferative disease can be, for example, hyperplasias, endometriosis, hypertrophic scars and keloids, proliferative diabetic retinopathy, glomerulonephritis, proliferative, pulmonary hypertension, rheumatoid arthritis, arteriovenous malformations, atherosclerotic plaques, coronary artery disease, delayed wound healing, hemophilic joints, nonunion fractures, Osler-Weber syndrome, psoriasis, pyogenic granuloma, scleroderma, tracoma, menorrhagia, vascular adhesions, and papillomas, and (d) the fibrotic disease can be, for example, hepatic fibrosis, pulmonary fibrosis and retroperitoneal fibrosis.

The animal can be any patient or subject in need of treatment or diagnosis. In preferred embodiments, the animal is a mammal. In particularly preferred embodiments, the animal is a human. In other embodiments, the animal can be a mouse, rat, rabbit, cat, dog, pig, sheep, horse, cow, or a non-human primate.

The invention also provides a method for inhibition of SPARC activity using neutralizing antibody against SPARC, e.g., a suitable SPARC binding antibody. A neutralizing antibody has the ability to block the interaction of SPARC with its effectors in vivo, for example, the interaction of SPARC with cell surface component or the binding of SPARC to its natural ligands such as albumin, growth factors, and Ca²⁺. The invention provides a method for delivering a chemotherapeutic agent to a tumor in a mammal. The methods comprise administering to a human or other animal a therapeutically effective amount of a pharmaceutical composition, wherein the pharmaceutical composition comprises the chemotherapeutic agent coupled to a suitable SPARC binding antibody and a pharmaceutically acceptable carrier. Descriptions of the chemotherapeutic agents, animals, and components thereof, set forth herein in connection with other embodiments of the invention also are applicable to those same aspects of the aforesaid method of delivering a chemotherapeutic agent to a tumor.

The types of tumor to be detected, whose response to chemotherapy can be predicted or determined, which can be treated in accordance with the invention are generally those found in humans and other mammals. The tumors can be the result of inoculation as well, such as in laboratory animals. Many types and forms of tumors are encountered in human and other animal conditions, and there is no intention to limit the application of the methods of the present to any particular tumor type or variety. Tumors, as is known, include an abnormal mass of tissue that results from uncontrolled and progressive cell division, and is also typically known as a “neoplasm.” The inventive methods are useful for tumor cells and associated stromal cells, solid tumors and tumors associated with soft tissue, such as, soft tissue sarcoma, for example, in a human.

The tumor or cancer can be located in the oral cavity and pharynx, the digestive system, the respiratory system, bones and joints (e.g., bony metastases), soft tissue, the skin (e.g., melanoma), breast, the genital system, the urinary system, the eye and orbit, the brain and central nervous system (e.g., glioma), or the endocrine system (e.g., thyroid) and is not necessarily limited to the primary tumor or cancer. Tissues associated with the oral cavity include, but are not limited to, the tongue and tissues of the mouth. Cancer can arise in tissues of the digestive system including, for example, the esophagus, stomach, small intestine, colon, rectum, anus, liver, gall bladder, and pancreas. Cancers of the respiratory system can affect the larynx, lung, and bronchus and include, for example, small cell and non-small cell lung carcinoma. Tumors can arise in the uterine cervix, uterine corpus, ovary vulva, vagina, prostate, testis, and penis, which make up the male and female genital systems, and the urinary bladder, kidney, renal pelvis, and ureter, which comprise the urinary system. The tumor or cancer can be located in the head and/or neck (e.g., laryngeal cancer and parathyroid cancer). The tumor or cancer also can be located in the hematopoietic system or lymphoid system, and include, for example, lymphoma (e.g., Hodgkin's disease and Non-Hodgkin's lymphoma), multiple myeloma, or leukemia (e.g., acute lymphocytic leukemia, chronic lymphocytic leukemia, acute myeloid leukemia, chronic myeloid leukemia, and the like). Preferably, the tumor is located in the bladder, liver, ovary, kidney, gut, brain, or breast.

In other embodiments, the invention provide a methods for delivering a pharmaceutically active agent by way of a SPARC binding antibody to a site of disease that is characterized by overexpression of SPARC. Such diseases include abnormal conditions of proliferation, tissue remodeling, hyperplasia, and exaggerated wound healing in bodily tissue (e.g., soft tissue, connective tissue, bone, solid organs, blood vessel and the like). Examples of diseases that are treatable or can be diagnosed by administering a pharmaceutical composition comprising a therapeutic agent coupled to a suitable SPARC antibody, include cancer, diabetic or other retinopathy, inflammation, arthritis, restenosis in blood vessels, artificial blood vessel grafts, or intravascular devices, and the like. Descriptions of the chemotherapeutic agents, tumors, animals, and components thereof, set forth herein in connection with other embodiments of the invention also are applicable to those same aspects of the aforesaid method of delivering a pharmaceutically active agent.

In other embodiments, the inventive methods comprise administering to a mammal a therapeutically effective amount of a pharmaceutical composition comprising a liposome bound or albumin bound chemotherapeutic agent wherein the liposome or albumin is coupled to a suitable disease targeting SPARC binding antibody. The chemotherapeutic agent can be coupled to the SPARC binding antibody using any suitable method. Preferably, the chemotherapeutic agent is chemically coupled to the compound via covalent bonds including, for example, disulfide bonds.

One or more doses of one or more chemotherapeutic agents, such as those described above, can also be administered according to the inventive methods. The type and number of chemotherapeutic agents used in the inventive method will depend on the standard chemotherapeutic regimen for a particular tumor type. In other words, while a particular cancer can be treated routinely with a single chemotherapeutic agent, another can be treated routinely with a combination of chemotherapeutic agents. Methods for coupling or conjugation of suitable therapeutics, chemotherapeutics, radionuclides, etc. to antibodies or fragments thereof are well described in the art. The following examples further illustrate the invention but, of course, should not be construed as in any way limiting its scope.

Methods in accordance with the invention include, e.g., combination therapies wherein the animal is also undergoing one or more cancer therapies selected from the group consisting of surgery, chemotherapy, radiotherapy, thermotherapy, immunotherapy, hormone therapy and laser therapy. The terms “co-administration” and “combination therapy” refer to administering to a subject two or more therapeutically active agents. The agents can be contained in a single pharmaceutical composition and be administered at the same time, or the agents can be contained in separate formulation and administered serially to a subject. So long as the two agents can be detected in the subject at the same time, the two agents are said to be co-administered.

Combination therapies contemplated in the present invention include, but are not limited to antibody administration, vaccine administration, administration of cytotoxic agents, natural amino acid polypeptides, nucleic acids, nucleotide analogues, and biologic response modifiers. Two or more combined compounds may be used together or sequentially. Examples of chemotherapeutic agents include alkylating agents, antimetabolites, natural products, hormones and antagonists, and miscellaneous agents. Examples of alkylating agents include nitrogen mustards such as mechlorethamine, cyclophosphamide, ifosfamide, melphalan (L-sarcolysin) and chlorambucil; ethylenimines and methylmelamines such as hexamethylmelamine and thiotepa; alkyl sulfonates such as busulfan; nitrosoureas such as carmustine (BCNU), semustine (methyl-CCNU), lomustine (CCNU) and streptozocin (streptozotocin); DNA synthesis antagonists such as estramustine phosphate; and triazines such as dacarbazine (DTIC, dimethyl-triazenoimidazolecarboxamide) and temozolomide. Examples of antimetabolites include folic acid analogs such as methotrexate (amethopterin); pyrimidine analogs such as fluorouracin (5-fluorouracil, 5-FU, 5FU), floxuridine (fluorodeoxyuridine, FUdR), cytarabine (cytosine arabinoside) and gemcitabine; purine analogs such as mercaptopurine (6-niercaptopurine, 6-MP), thioguanine (6-thioguanine, TG) and pentostatin (2′-deoxycoformycin, deoxycoformycin), cladribine and fludarabine; and topoisomerase inhibitors such as amsacrine. Examples of natural products include vinca alkaloids such as vinblastine (VLB) and vincristine; taxanes such as paclitaxel (Abraxane®) and docetaxel (Taxotere®); epipodophyllotoxins such as etoposide and teniposide; camptothecins such as topotecan and irinotecan; antibiotics such as dactinomycin (actinomycin D), daunorubicin (daunomycin, rubidomycin), doxorubicin, bleomycin, mitomycin (mitomycin C), idarubicin, epirubicin; enzymes such as L-asparaginase; and biological response modifiers such as interferon alpha and interleukin 2. Examples of hormones and antagonists include luteinising releasing hormone agonists such as buserelin; adrenocorticosteroids such as prednisone and related preparations; progestins such as hydroxyprogesterone caproate, medroxyprogesterone acetate and megestrol acetate; estrogens such as diethylstilbestrol and ethinyl estradiol and related preparations; estrogen antagonists such as tamoxifen and anastrozole; androgens such as testosterone propionate and fluoxymesterone and related preparations; androgen antagonists such as flutamide and bicalutamide; and gonadotropin-releasing hormone analogs such as leuprolide. Examples of miscellaneous agents include thalidomide; platinum coordination complexes such as cisplatin (czs-DDP), oxaliplatin and carboplatin; anthracenediones such as mitoxantrone; substituted ureas such as hydroxyurea; methylhydrazine derivatives such as procarbazine (N-methylhydrazine, MIH); adrenocortical suppressants such as mitotane (o,p′-DDD) and aminoglutethimide; RXR agonists such as bexarotene; and tyrosine kinase inhibitors such as imatinib.

Compositions featured in the methods of the present invention can be administered in a single dose or in multiple doses. Where the administration of the antibodies by infusion, the infusion can be a single sustained dose or can be delivered by multiple infusions. Injection of the agent can be directly into the tissue at or near the site of aberrant target gene expression. Multiple injections of the agent can be made into the tissue at or near the site.

Dosage levels on the order of about 1 ug/kg to 100 mg/kg of body weight per administration are useful in the treatment of a disease. In regard to dosage, an antibody can be administered at a unit dose less than about 75 mg per kg of bodyweight, or less than about 70, 60, 50, 40, 30, 20, 10, 5, 2, 1, 0.5, 0.1, 0.05, 0.01, 0.005, 0.001, or 0.0005 mg per kg of bodyweight, and less than 200 nmol of antibody per kg of bodyweight, or less than 1500, 750, 300, 150, 75, 15, 7.5, 1.5, 0.75, 0.15, 0.075, 0.015, 0.0075, 0.0015, 0.00075, 0.00015 nmol of antibody per kg of bodyweight. The unit dose, for example, can be administered by injection (e.g., intravenous or intramuscular, intrathecally, or directly into an organ), inhalation, or a topical application.

One skilled in the art can also readily determine an appropriate dosage regimen for administering the antibody of the invention to a given subject. For example, the SPARC-binding antibody composition can be administered to the subject once, as a single injection or deposition at or near the site of SPARC expression. Compositions of the present invention can be administered daily, semi-weekly, weekly, bi-weekly, semi-monthly, monthly, bi-monthly, or at the discretion of the clinician. In some embodiments, the compositions are administered once or twice daily to a subject for a period of from about three to about twenty-eight days, more preferably from about seven to about ten days. In further embodiments, the unit dose is administered less frequently than once a day, e.g., less than every 2, 4, 8 or 30 days. In other embodiments, the unit dose is not administered with a frequency (e.g., not a regular frequency).

Where a dosage regimen comprises multiple administrations, it is understood that the effective amount of SPARC-binding antibody composition administered to the subject can include the total amount of antibody administered over the entire dosage regimen. One skilled in the art will appreciate that the exact individual dosages may be adjusted somewhat depending on a variety of factors, including the specific SPARC binding antibody composition being administered, the time of administration, the route of administration, the nature of the formulation, the rate of excretion, the particular disorder being treated, the severity of the disorder, the pharmacodynamics of the oligonucleotide agent, and the age, sex, weight, and general health of the patient. Wide variations in the necessary dosage level are to be expected in view of the differing efficiencies of the various routes of administration.

The effective dose can be administered in a single dose or in two or more doses, as desired or considered appropriate under the specific circumstances. If desired to facilitate repeated or frequent infusions, implantation of a delivery device, e.g., a pump, semi-permanent stent (e.g., intravenous, intraperitoneal, intracisternal or intracapsular), or reservoir may be advisable. Following successful treatment, it may be desirable to have the patient undergo maintenance therapy to prevent the recurrence of the disease state. The concentration of the antibody composition is an amount sufficient to be effective in treating or preventing a disorder or to regulate a physiological condition in humans. The concentration or amount of antibody administered will depend on the parameters determined for the agent and the method of administration.

Certain factors may influence the dosage required to effectively treat a subject, including but not limited to the severity of the disease or disorder, previous treatments, the general health and/or age of the subject, and other diseases present. It will also be appreciated that the effective dosage of the antibody used for treatment may increase or decrease over the course of a particular treatment. Changes in dosage may result and become apparent from the results of diagnostic assays. For example, the subject can be monitored after administering an antibody composition. Based on information from the monitoring, an additional amount of the antibody composition can be administered. Persons of ordinary skill can easily determine optimum dosages, dosing methodologies and repetition rates.

### Example 1

This Example demonstrates the preparation of a series of antibodies capable of binding to human SPARC.

Twelve mouse-derived anti-human SPARC antibodies were commercially generated using a conventional hybridoma approach using mouse strain RBF/DnJ.

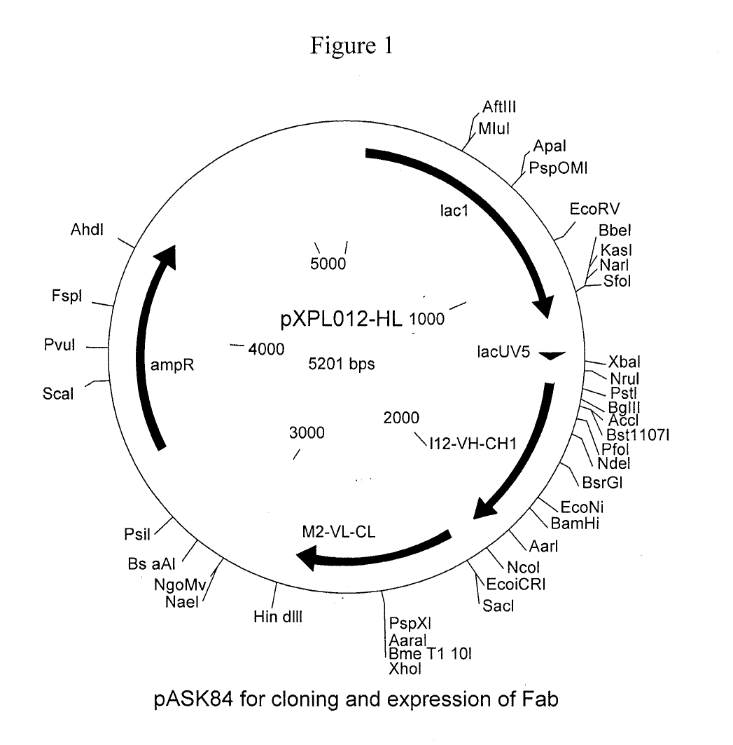

A pASK84 expression vector (FIG. 1) was used to express the Fab regions of the resulting antibodies, designated Imm1 through Imm12. The Fab regions were targeted to the periplasm where they were collected and subsequently purified via activity chromatography on a protein A sepharose column. Identity was verified by Western blot and SPARC binding activity was verified by ELISA.

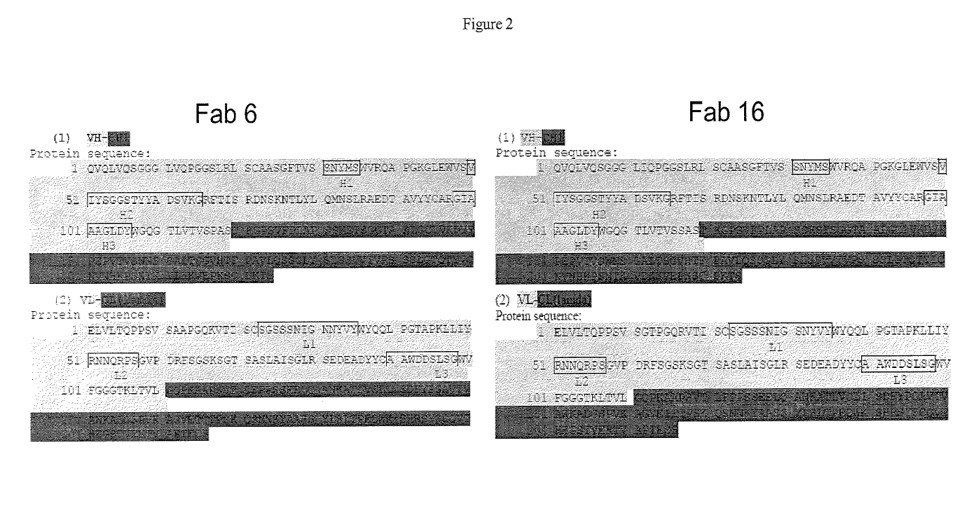

Imm13 and Imm14 are fully human anti-human SPARC antibodies which were generated using a human phage display library. SPARC was panned against the commercial human Fab phage display library HuFabL® (Creative Biolabs, Shirley, N.Y.). Two Fab sequences of interest were identified: Fab6 (SEQ ID NO 15) and Fab16 (SEQ ID NO 16), as shown in FIG. 2. SPARC binding activity was verified by ELISA for these two Fab molecules.

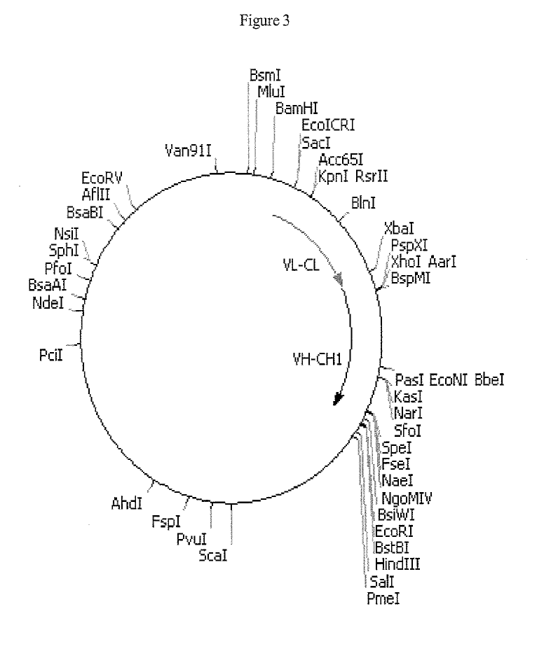

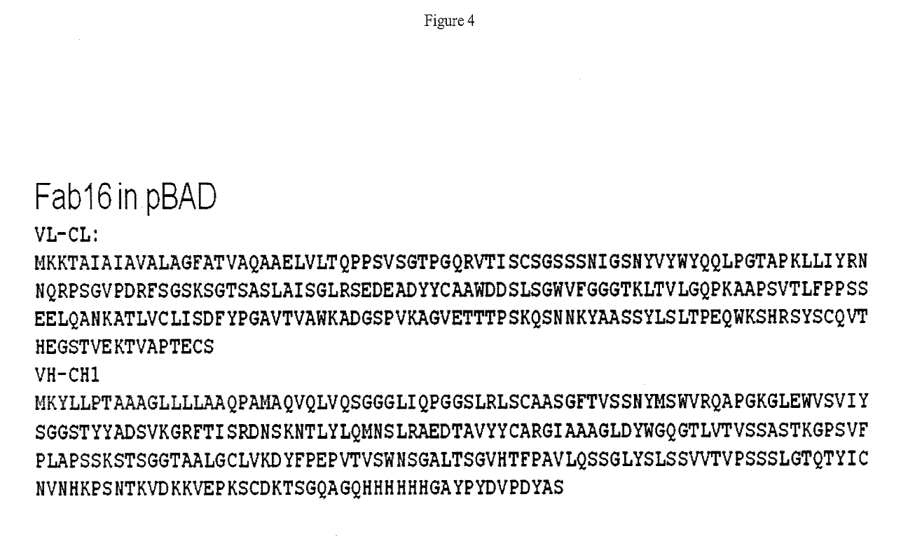

These Fab regions were cloned into the pBAD vector (FIG. 3) and were expressed and purified in bacteria. The Fab proteins expressed by the pBAD vector were isolated from the periplasmic fraction of lysed bacteria, with sequences provided at FIG. 4. The identities of the Fab regions obtained from the periplasmic fraction were verified by SDS page. The Fab proteins were purified to homogeneity via activity chromatography on a protein A sepharose column.

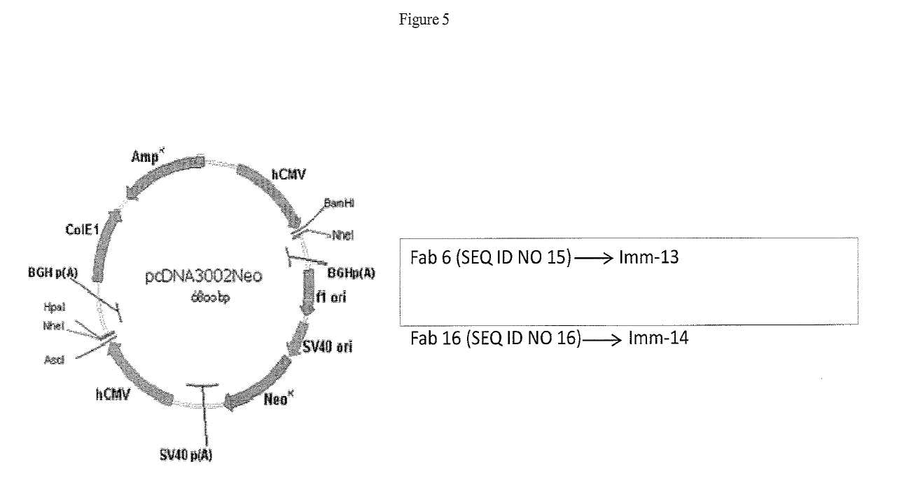

In order to create fully human SPARC binding antibodies, the genes for Fab6 and Fab16 were cloned and expressed via the pcDNA3002Neo Vector (Invitrogen, Carlsbad, Calif.) (FIG. 5). The resultant antibodies were purified and their identities were verified by gel electrophoresis and N-terminal analysis. The fully human antibody created from Fab6 was designated Imm13 and the fully human antibody created from Fab16 was designated Imm14.

After they were generated according to the foregoing methods, Imm1 through Imm14 antibodies were characterized according to isotype by utilizing a commercial mouse isotyping test kit (AbD Serotec, Raleigh, N.C.). The results are presented in Table 1.

TABLE 1

| | Abraxis | |
|---|---|---|
| Clone Number | Name | Isotype |
| 16 | Imm1 | IgG1 (κ) |
| 38 | Imm2 | IgG1, 2b (κ) |
| 39 | Imm3 | IgG1, 2b (κ) |
| 43 | Imm4 | IgG1 (κ) |
| 47 | Imm5 | IgG2a (κ) |
| 49 | Imm6 | IgG1 (κ) |
| 55 | Imm7 | IgG2a (κ) |
| 58 | Imm8 | IgG2b (κ) |
| 62 | Imm9 | IgG1 (κ) |
| 66 | Imm10 | IgG1 (κ) |
| 70 | Imm11 | IgG1 (κ) |
| 71 | Imm12 | IgG1 (κ) |
| F6 | Imm13 | IgG1 (κ) |
| F16 | Imm14 | IgG1 (κ) |

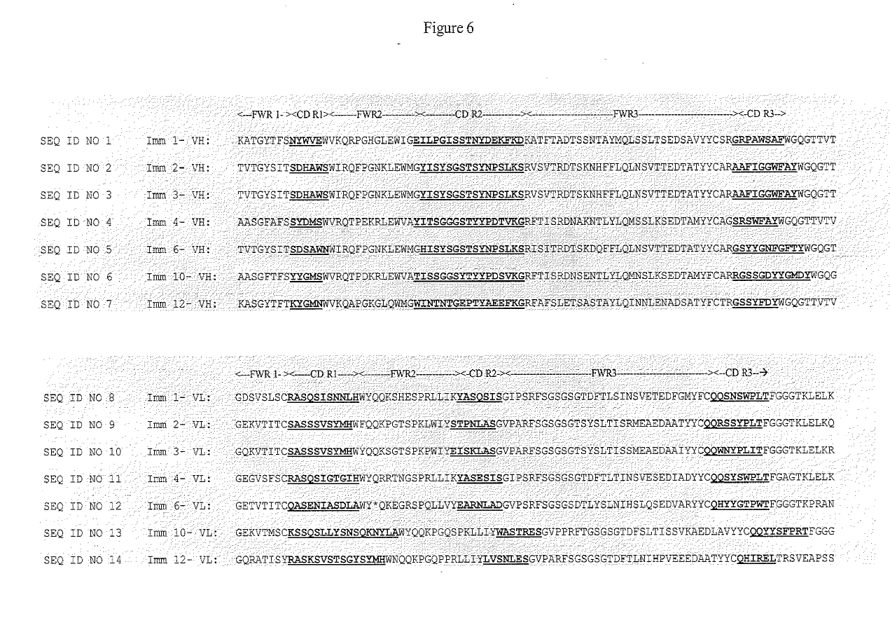

The sequences for the variable complimentary determining regions for selected Imm series antibodies, including Imm12, are presented in FIG. 6. The clones in Table 1, Imm1 through Imm14, will be deposited at a suitable depository, such as the ATCC.

### Example 2

This Example demonstrates the use of ELISA assays to characterize the SPARC binding of the Imm series antibodies.

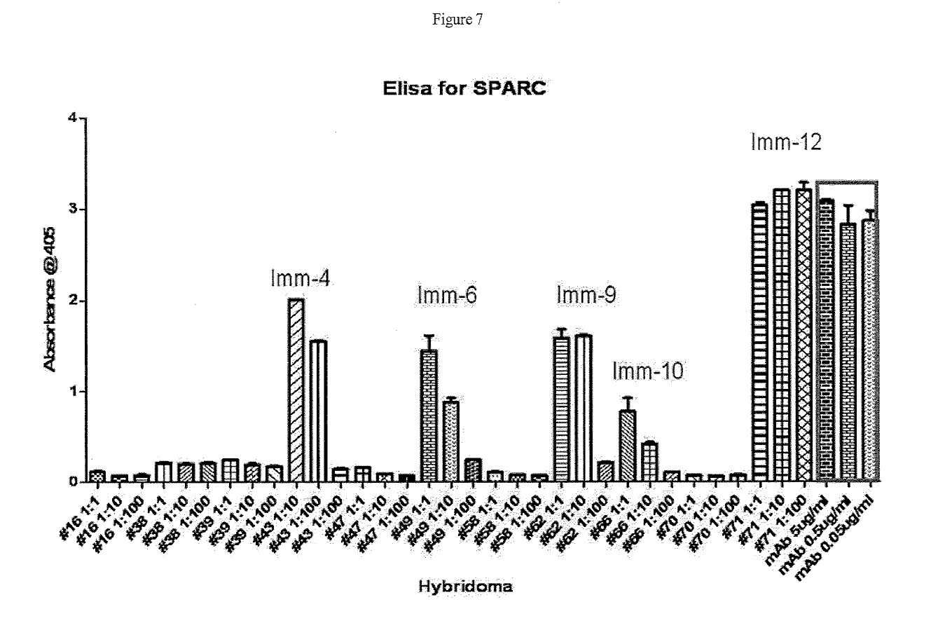

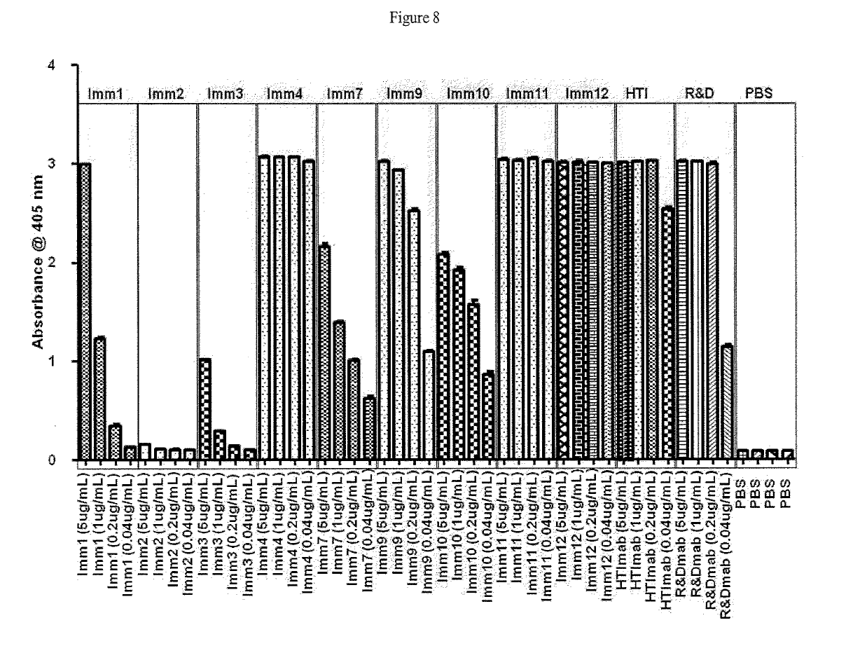

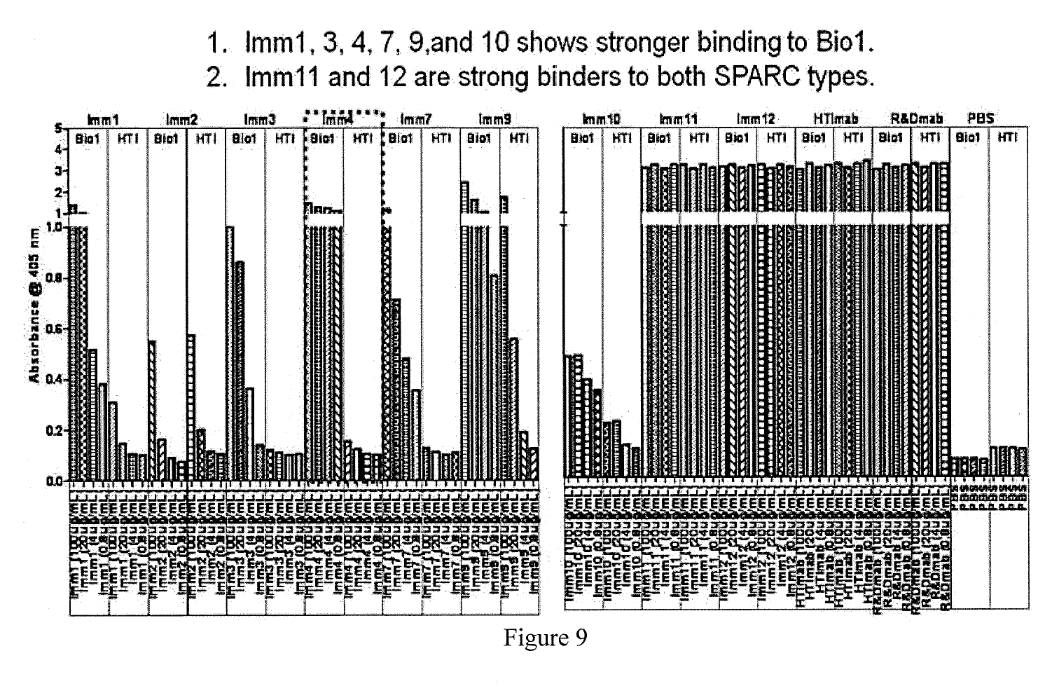

The ability of Imm1 through Imm12 (the mouse-derived anti-human SPARC antibodies) to bind recombinant human SPARC (Bio 1-SPARC) was characterized by multiple ELISA assays performed at various stages of purification. FIG. 7 presents the results of an ELISA assay performed on a serial dilution (1:1, 1:10, and 1:100) of antibody supernatants prior to purification. In this assay, Imm4, Imm6, Imm9, Imm10 and Imm12 exhibited the highest Bio1-SPARC binding, with Imm12 exhibiting the highest binding overall. Another ELISA assay was performed with the purified antibodies (FIG. 8) at concentrations of 0.04 μg/mL, 0.2 μg/mL, 1 μg/mL, and 5 μg/mL. The binding of the purified antibodies was generally improved over the unpurified supernatants. In this assay, Imm4, Imm9, Imm11 and Imm12 exhibited the highest Bio1-SPARC binding. An additional ELISA was performed to compare the binding of the mouse derived Immseries antibodies to two different varieties of SPARC: Bio1-SPARC, and human platelet SPARC (HTI-SPARC) (FIG. 9). In this assay, Imm4 and Imm9 were both found to bind Bio1-SPARC significantly better than HTI-SPARC. Imm11 and Imm12 bind both varieties of human SPARC equally well.

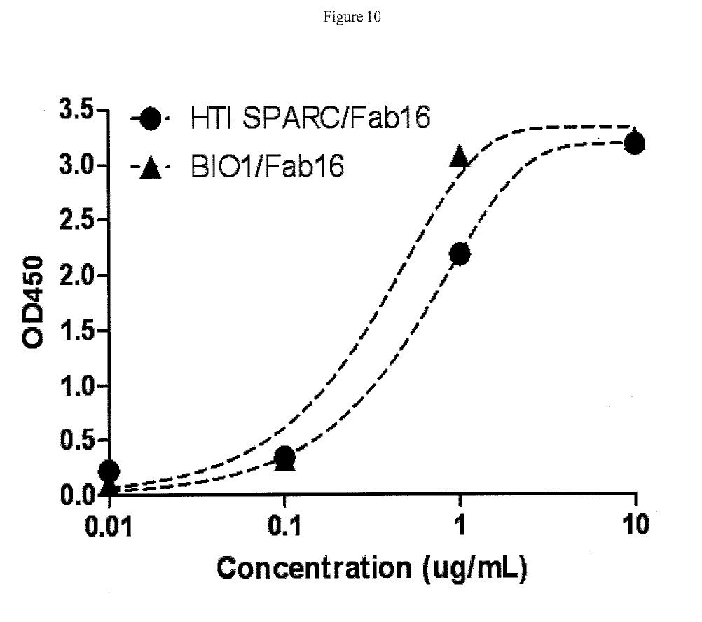

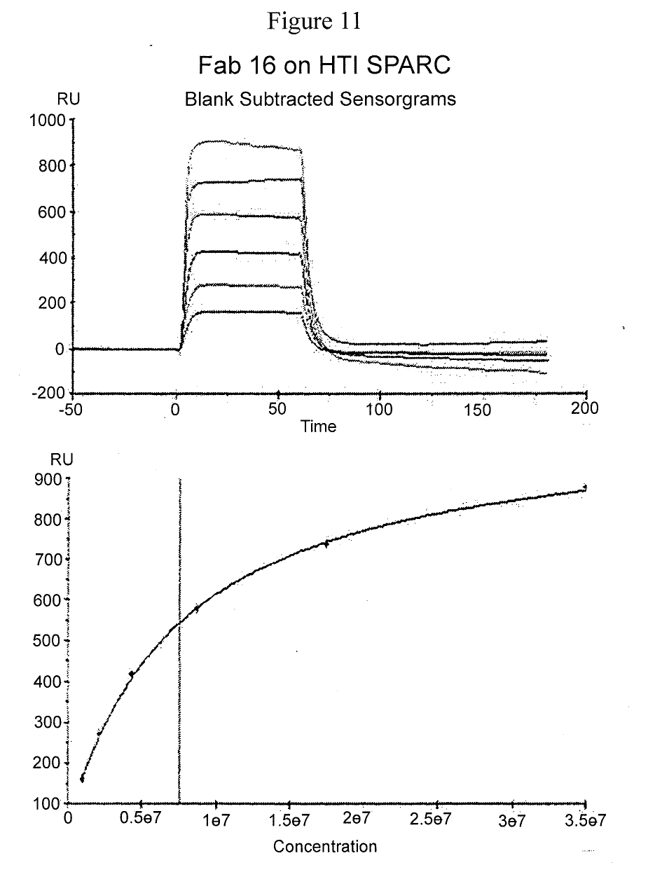

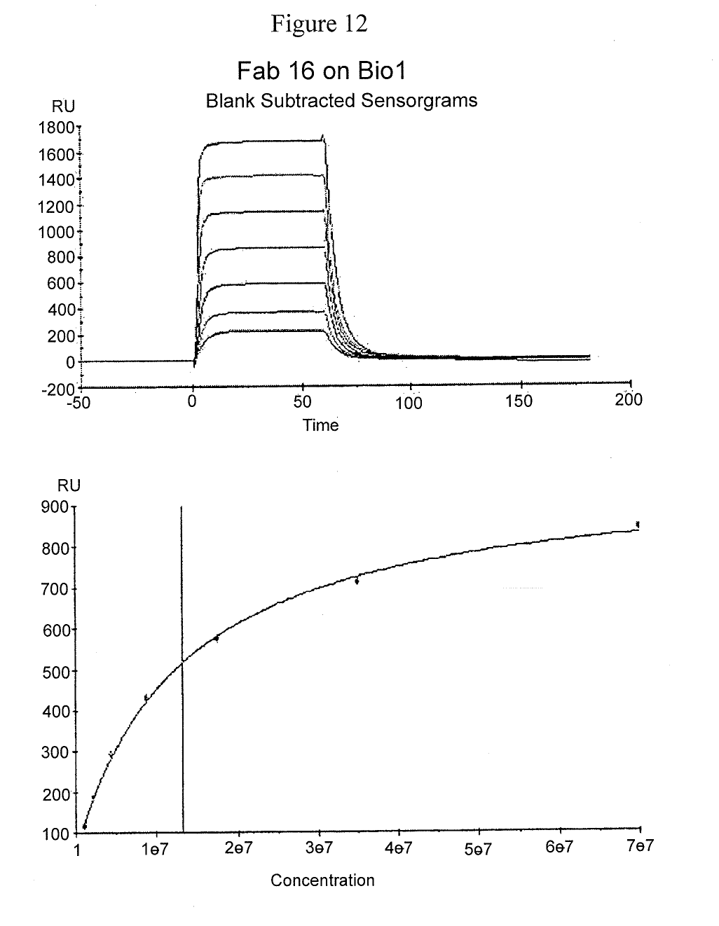

ELISA assays were also used to characterize the SPARC binding of the fully human SPARC binding antibodies, Imm13 and Imm14. For example, according to a protein ELISA assay (FIG. 10), Fab16 (the Fab region of Imm14) binds HTI-SPARC with a K_{D} of 11 nM and binds Bio1-SPARC with a K_{D} of 7 nM. Surface plasmon resonance binding assays, performed on the Biacore 3000® (GE/Biacore International AB, Uppsala, Sweden), tested the binding of Fab 16 to both varieties of SPARC immobilized on a sensorchip (FIGS. 11 and 12). These assays resulted in K_{D} values of 76.2 nM for HTI SPARC and 132 nM for Bio1-SPARC.

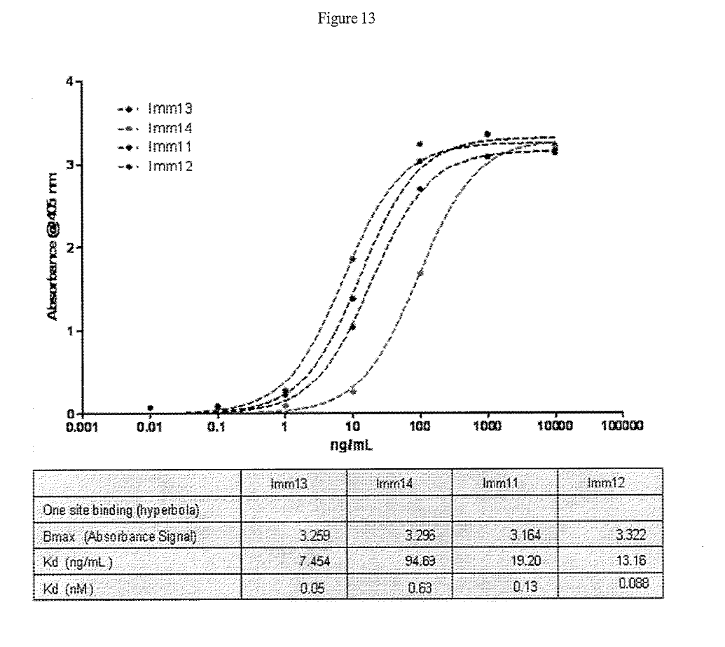

An ELISA assay was also performed to directly compare the SPARC binding capabilities of selected mouse-derived anti human SPARC antibodies, Imm11 and Imm12, to the fully human Imm13 and Imm14, the results of which are presented in FIG. 13. The results indicate that Imm13 has a higher affinity for SPARC than both of the mouse derived antibodies, while Imm14 has a lower affinity.

This example demonstrates that certain of the Imm series antibodies bind, in vitro, to both recombinant human SPARC and human platelet SPARC in binding assays.

### Example 3

This example demonstrates the analysis of the epitopes to which the Imm series antibodies bind.

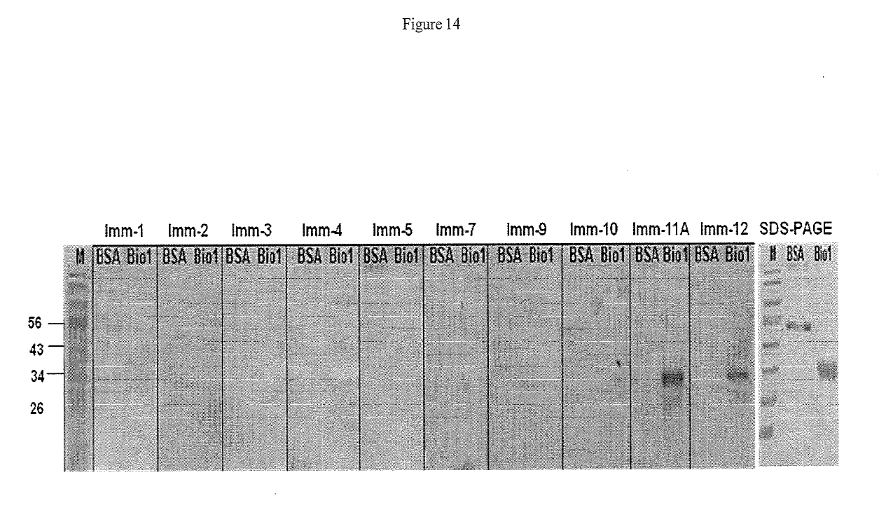

Western blotting was used to determine whether the Imm series antibodies bind to linear or conformational epitopes. In this analysis, SPARC protein was run on a polyacrylamide gel in the presence of SDS. Accordingly, the SPARC protein on the gel was in its denatured form. The Imm series antibodies were used as primary antibodies and were then probed with goat anti-mouse IgG. BSA was used as a negative control. The results of the assay, shown at FIG. 14, show binding of Imm11 and Imm12 to SPARC, while Imm1 through Imm11 did not bind denatured SPARC. In a subsequent test, Imm14 and mHTI were also found to bind denatured SPARC (data not shown).

These results show that Imm12, Imm14, and mHTI are capable of binding SPARC based on linear, or primary, epitopes.

### Example 4

This example discusses results of an in vivo assay examining the effect of certain antibodies on survival in nude mice challenged with LL/2 Lewis Lung Carcinoma.

C57BL male mice (approx. 6 weeks old) were weighed, and then injected intravenously with approximately 1×10⁶ cells (approximately 0.1 mL) of LL/2 (Lewis Lung Carcinoma) with 25-gauge needle. A minimum of 10 animals was used per group. Twice weekly for 4 weeks, each animal in all groups received 200 μg/mouse of a test antibody or mouse IgG. These test articles/antibodies were dosed via intra-abdominal injection into the peritoneal cavity with a 25-27 gauge needle. The first dose was administered within 30 minutes of tumor cell injection.

Animals were weighed prior to tumor cell injection, and twice weekly prior to dosing. All animals were examined twice daily for mortality/morbidity. At the end of the experiment, the mice were killed, and primary tumors and lungs were removed, fixed, and embedded. Euthanized animals and any animals found dead prior to rigor mortis were necropsied. Plasma and lungs from all animals were collected and weighed at termination. Lungs were infused with and then immersed in 10% Neutral buffered formalin. Tissues were stored for additional gross and histopathologic analysis.

Animals were administered either mHTI, Imm12, or Imm14. A negative control group was administered mIgG. The antibodies were formulated in PBS and were administered in at dose of 200 μg/mouse, twice weekly, for four weeks. The survival of the animals was then recorded over twenty days.

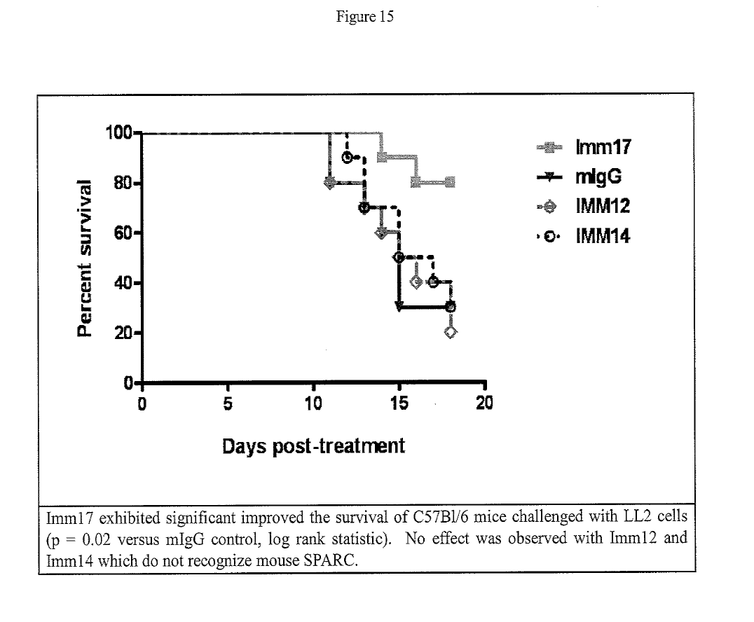

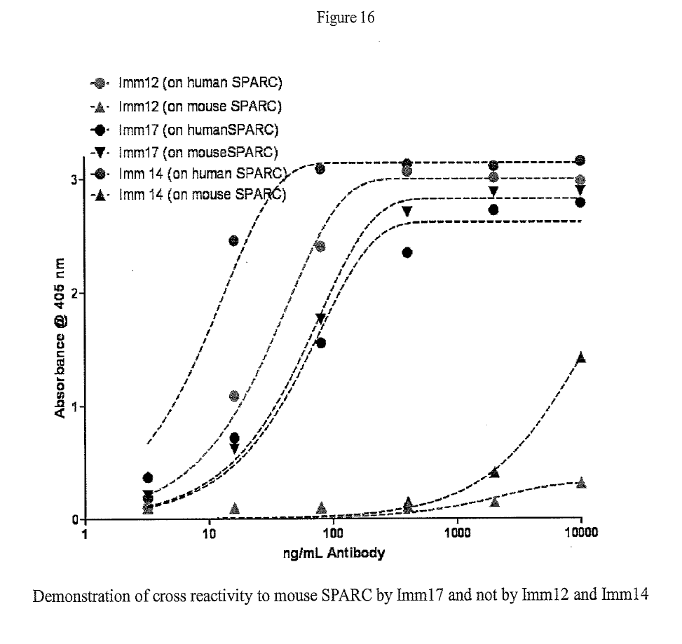

A higher percentage of animals treated with mHTI survived at the various time points than did animals treated with Imm12 or Imm14. As shown in FIG. 15, only mHTI was effective in inhibiting lung metastasis in the mouse, p=0.02 versus mouse control IgG, log rank statistic. As expected, Imm12 and Imm14 which do not recognize mouse SPARC, but do recognize human SPARC, did not inhibit lung LL/2 metastasis. As shown in FIG. 16, Imm12 and Imm14 specifically bind to human SPARC and not mouse SPARC, while mHTI is capable of binding both mouse and human SPARC.

These results show that specific inhibition of mouse SPARC by mHTI resulted in inhibition of the colonization and/or growth of syngeneic LL/2 cells in the mouse and indicate that mHTI, or a humanized version thereof, may be useful in treating cancer.

### Example 5

This example describes a study consisting of two parallel phase II clinical trials (cohorts) to assess the anti-tumor activity and safety profile of the combination of carboplatin and nab-paclitaxel (Abraxane®, also designated ABI-007) in patients with unresectable stage 1V malignant melanoma.

Cohort 1 consisted of patients that were previously treated with chemotherapy, and cohort 2 consisted of patients that were newly diagnosed and chemotherapy naïve.

The data presented herein is from a multi-institution cooperative group study conducted through the North Central Cancer Treatment Group (NCCTG). This study was approved by the institutional review boards of all participating institutions. Written informed consent was obtained from all participants. Eligible patients were 18 years of age or older, with unresectable, histologically confirmed, stage 1V melanoma. Additional eligibility criteria included a measurable disease as defined by the Response Evaluation Criteria in Solid Tumors (RECIST), an Eastern Cooperative Oncology Group (ECOG) performance status (PS) of 0-2, a life expectancy of 3 months or greater, adequate hematologic and hepatic function, 4 weeks or more elapsed since last chemotherapy treatment (cohort 1 only), radiation therapy, or immunotherapy. Exclusion criteria included: any prior treatment with platinum or taxanes (cohorts 1 & 2), any prior chemotherapy for metastatic disease (cohort 2), active infection, New York Heart Association Class III or IV, peripheral neuropathy of grade 2 or higher; other malignancy in the last 5 years (except for non-melanomatous skin cancer or carcinoma in situ of the cervix) or untreated metastatic melanoma to the brain or progression of brain metastasis within 3 months of study entry. Women who were pregnant or breast feeding were not enrolled.

Eligible patients (both cohorts) were treated with 100 mg/m² of nab-paclitaxel by intravenous infusion over 30 minutes followed by carboplatin (CBDCA) with a target AUC of 2 (by Calvert formula with Cockroft and Gault Equation and actual body weight) over 30 minutes on days 1, 8, and 15 of a 28 day cycle, for a maximum of 8 cycles. If patients did not develop excessive toxicity or progressive disease, treatment beyond 8 cycles was at the discretion of the treating physician. Within 14 days of registration, patients underwent a complete physical exam, assessment of ECOG PS, complete blood cell count (CBC), comprehensive metabolic panel including lactic dehydrogenase (LDH), and a tumor assessment by conventional CT or MRI or spiral CT. Prior to each cycle of treatment, patients underwent a physical exam, toxicity assessments, and blood draws for hematologic and chemistry groups. Tumor status was assessed every 8 weeks until progression using RECIST criteria. On day 1 of each treatment cycle, treatment was withheld if absolute neutrophil count (ANC) was less than 1,500/mm³, platelet count (PLT) was less than 100,000/mm³, the patient developed a grade 2 or higher AST neuropathy, or other grade 3 or higher non-hematologic toxicity. When patients had recovered from these toxicities, treatment was re-started with a 20% dose reduction in both agents. On days 8 or 15 of each treatment cycle, treatment was omitted if: ANC was less than 1,000/mm³ or PLT was less than 100,000/mm³ or patient developed either a grade 2 or higher neuropathy or grade 3 or higher non-hematologic toxicity. Study treatment was terminated if toxicities did not recover to acceptable levels within 4 weeks and/or if patients required a third dose reduction due to toxicity. All patients received standard supportive care, including antiemetics, antibiotics, blood/platelet transfusions, erythropoietin and colony stimulating factors at the discretion of the treating physician.

Thirty five patients were accrued to Cohort 1, and 41 patients were accrued to Cohort 2 between Nov. 15, 2006 and Jul. 31, 2007 (Table 2). In Cohort 1 (PT) 1 patient canceled participation after signing a consent form but prior to the start of treatment. As such, the study Cohort 1 consists of 34 patients (67.6% male) who began study treatment. The median age at enrollment was 60 years (ages ranged from 28 to 84 years). In Cohort 2 (CN), 2 patients canceled participation after signing a consent form but prior to the start of treatment. As such, the study Cohort 2 consists of 39 patients (59.0% male) who began study treatment. The median age at enrollment was 59 years (ages ranged from 23 to 91 years).

For Cohort 1 the median number of cycles administered was 4 cycles (total: 135 cycles, range: 1-10). Twenty one patients (61.8%) were omitted from treatments on day 8 or 15 of treatment or had at least one dose reduction. This was primarily due to severe neutropenia, fatigue, and neuropathy. The main reason for study discontinuation was progression of disease (27 patients).

For Cohort 2, the median number of cycles administered was 4 cycles (total: 193 cycles, range: 1-25). Twenty five patients were omitted from treatments on day 8 or 15 of treatment or had at least one dose reduction, largely due to severe neutropenia and neuropathy. The primary reason for study discontinuation was progression ° f disease (27 patients).

The prognostic utility of plasma SPARC was evaluated by stratifying patients into “high” and “low” SPARC groups. As the median for plasma SPARC was 431 ng/ml, high SPARC group was defined as patients with plasma SPARC above 431 ng/ml. The breakdown of the patient population is shown in Table 2. With 1 exception, the results show that “high SPARC” patients tend to have worse progression free survival (PFS) and overall survival (OS) than their “low SPARC” counterparts, although only OS in the Prior Chemo group was found to be statistically significant (p=0.01).

TABLE 2

| | Median | P-value | N |
|---|---|---|---|
| Progression Free Survival | | | |
| Prior Chemotherapy group | | 0.21 | 31 |
| Low SPARC | 141 days | | 17 |
| High SPARC | 58 days | | 14 |
| No Prior Chemotherapy | | 0.47 | 35 |
| group | | | |
| Low SPARC | 122 days | | 16 |
| High SPARC | 167 days | | 19 |
| Overall Survival | | | |
| Prior Chemotherapy group | | 0.01 | 31 |
| Low SPARC | 378 days | | 17 |
| High SPARC | 206 days | | 14 |
| No Prior Chemotherapy | | 0.43 | 35 |
| group | | | |
| Low SPARC | 426 days | | 16 |
| High SPARC | 304 days | | 19 |

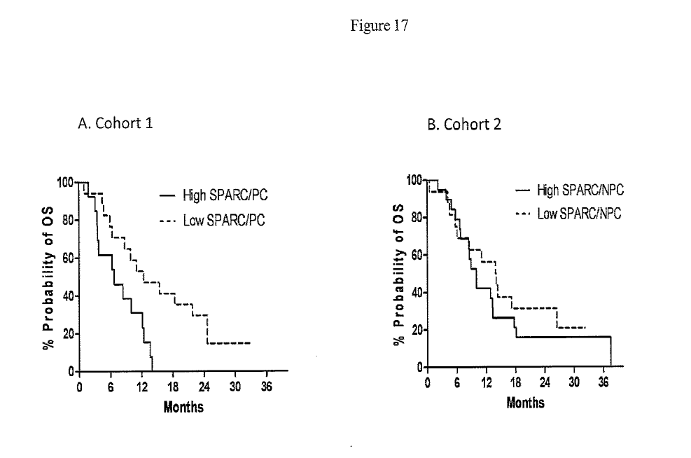

While the overall response rate differed significantly between the two cohorts (25.6% vs 8.8%), there was no difference in progression free survival or overall survival (FIG. 17 A-B). Overall, treatment was moderately well tolerated with the main toxicities being nausea, vomiting, peripheral neuropathy, and cytopenias (neutropenia, thrombocytopenia, leukopenia).

These results show that low circulating SPARC level was associated with improved overall survival. Additionally, the combination of nab-paclitaxel and carboplatin is a feasible therapeutic option for patients with metastatic melanoma who are either previously treated or chemotherapy naïve.

### Example 6

This example describes the evaluation of plasma SPARC concentration in samples derived from metastatic melanoma patients and healthy individuals.

ELISA plates were coated with 2.5 μg/ml SPARC binding polyclonal antibody (R&D Biosystems, Minneapolis, Minn.) in 50 mM carbonate buffer overnight at 4° C. Plates were washed 4 times with PBS/0.1% Tween 20 (PBST) and blocked for 2 hours at room temperature (RT) with casein blocking/dilution buffer (Thermo Fisher Scientific Inc., IL). For the generation of a SPARC standard curve, known concentrations of human platelet SPARC protein (Hematologic Technologies, Essex junction, VT) was diluted in blocking/dilution buffer containing SPARC negative 10% pooled normal human heparin plasma (PNHP). Before testing, patient samples were diluted 1/10 in blocking/dilution buffer. After removal of the blocking solution and three washes with PBST, standards and diluted plasma samples were plated onto the ELISA plates at 100 μl/well in duplicates and incubated for 2 hours at room temperature (RT), followed by three additional washes with PBST. For detection of bound SPARC, 100 ul of 0.5 μg/ml biotinylated anti SPARC monoclonal antibody (R&D Biosystems, Minneapolis, Minn.) in blocking/dilution buffer was added and incubated for 1 hour at RT, followed by 3 PBST washes. This was followed by 100 ul/well of 1:20000 diluted Streptavidin-Horseradish peroxidase (HRP) was added and incubated for 1 h at RT. After three PBST washes, 100 ul of HRP-Substrate TMB (KPL #52-00-03) was added to each well and OD at 650 nm was monitored. The reaction was stopped for measurement at OD 0.6 to 0.8 with 2N sulfuric acid. The optical density of the wells was read on an ELISA plate reader (Molecular Devices; Sunnyvale, Calif.) at 450 nm within 30 minutes.

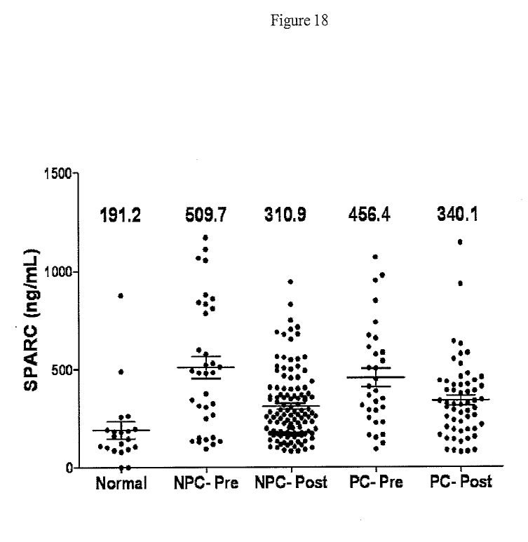

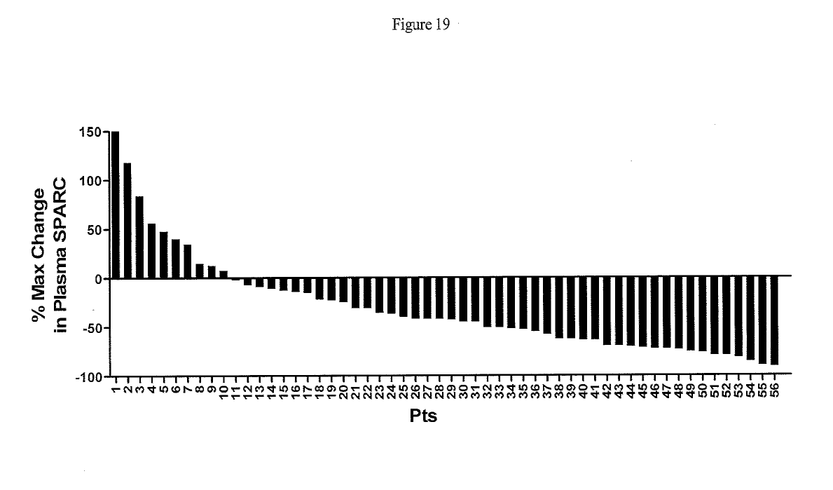

The results from a total of twenty samples derived from healthy individuals were compared to results from 65 cancer patient plasma samples as shown in FIG. 18. Analysis of the ELISA results revealed a statistically significant difference in the SPARC concentrations of both groups. SPARC levels in healthy individuals were determined at a median concentration of 192 ng/ml whereas the median plasma SPARC concentration in cancer patient samples was measured at 390 ng/ml (p value 0.0002) (FIG. 18). Additionally, treatment was followed with significant drop in plasma SPARC in the majority of the patients (FIG. 19).

These results demonstrate increased SPARC expression in metastatic melanoma patients and could be positively correlated with tumor burden.

### Example 7

This example demonstrates the preparation of a SPARC microenvironment signature (SMS).

A series of antibodies against SPARC were evaluated for their binding characteristics in a range of normal and tumor tissues. The SPARC expression pattern, as determined by immunostaining, in various components of tumors was determined including the SPARC expression levels in tumor cells, blood vessels, fibroblast, stroma, inflammatory cells, and the adjacent normal tissues. Two antibodies were identified with differential affinity for SPARC and were employed in follow up studies. Specifically, the pattern of staining was determined using a monoclonal antibody (“antibody M”) (SPARC monoclonal antibody (R&D Systems, Minneapolis, Minn.), catalog #MAB941 Lot # ECH045011 diluted 1:100 in a tris based diluent) and a polyclonal antibody (“antibody P”) (SPARC polyclonal antibody (R&D Systems, Minneapolis, Minn., catalog #AF941 Lot # EWN04 diluted 1:50 in a tris based diluents).

Histologic sections of tumors were prepared on slides and stained using a standard immunostaining protocol. Briefly, tissue cores from formalin-fixed, paraffin-embedded tumor blocks (2 cores from the most representative areas per block) were arrayed (Beecher Instruments, Silver Spring, Md.) to create a tissue microarray of cores measuring 2.0 mm each and were placed on positively charged slides. Slides with specimens were then placed in a 60° C. oven for 1 hour, cooled, deparaffinized, and rehydrated through xylenes and graded ethanol solutions to water. All slides were stained using automated staining equipment (Dako Cytomation Autostainer, Dako, Carpinteria, Calif.).

All slides were quenched for 5 minutes in a 3% hydrogen peroxide solution in water to block for endogenous peroxidase. After a buffer rinse, slides were incubated with antibody M or a negative control reagent for 30 minutes. A mouse horseradish peroxidase polymer kit (Mouse MACH 3 HRP Polymer Kit, Biocare Medical, Concord, Calif.) was incubated for 20 minutes per reagent. After another buffer rinse, DAB chromogen (Dako, Carpinteria, Calif.) was applied for 10 minutes. Hematoxylin was used to counterstain the slides. The same protocol was used for immunostaining specimens with antibody P, although an avidin-biotin detection kit (Biocare Medical, Concord, Calif.), incubated for 15 minutes per reagent, was used in place of the HRP detection kit.

Detailed pathological evaluation of SPARC expression in a series of tumors was performed by a board certified pathologist. The level of SPARC expression, as determined by immunohistochemistry, was scored for different tumor components. Scores were assigned to the level of SPARC expression on scale of 0-3, with 3 being the most positive score, as is commonly done in the art and well known to those of ordinary skill in the art.

The polyclonal antibody demonstrated preferential staining of SPARC in fibroblasts. While the monoclonal anybody preferably stained SPARC in tumor cells.

Logistic regression and proportional hazard were used to determine the correlation between response, progression-free survival (“PFS”) and overall survival (“OS”) to the SPARC pattern.

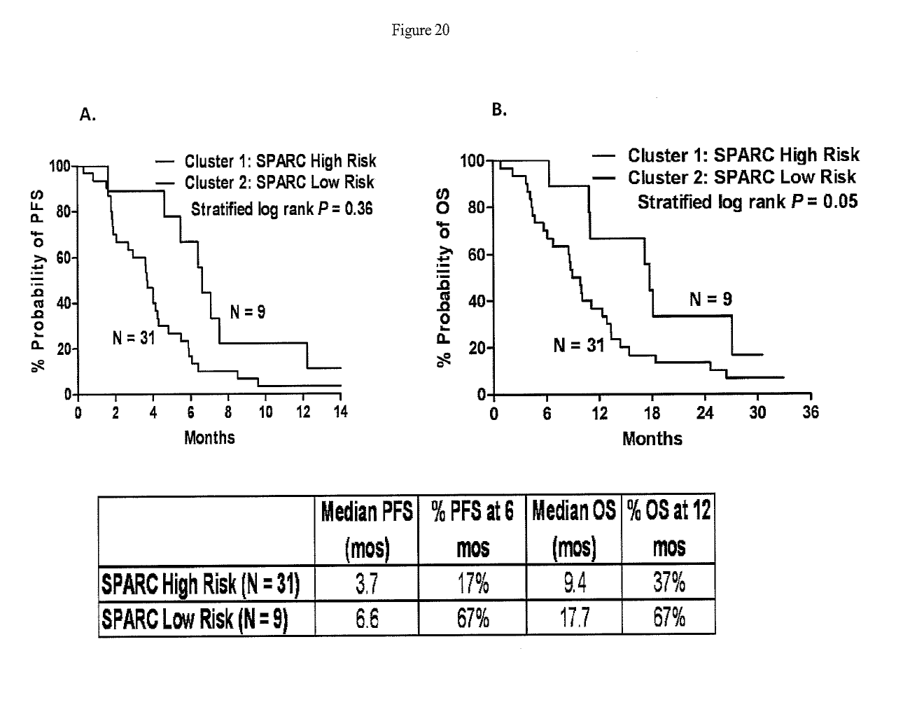

One of the tumor sets was a phase II trial of carboplatin and nab-paclitaxel (ABI-007) in patients with unresectable stage 1V melanoma. Specifically, nab-paclitaxel (100 mg/m²) and Carboplatin (AUC2) were administered on days 1, 8, and 15 of a 28 day cycle. SMS of the tumor biopsies were used to group the patients into two clusters, high risk (cluster 1) and low risk (cluster 2). As shown in Table 3 and FIGS. 20A-B, high risk and low risk SPARC signatures correlated with progression-free survival and overall survival.

TABLE 3

| | Median PFS | % PFS at 6 | Median OS | % OS at |
|---|---|---|---|---|
| | (months) | months | (months) | 12 months |
| Cluster 1 | 3.7 | 17% | 9.4 | 37% |
| (High Risk) | | | | |
| Cluster 2 (Low | 6.6 | 67% | 17.7 | 67% |
| Risk | | | | |

These results show that SPARC microenvironment signature alone can discriminate between low risk and high risk groups with respect to progression free survival and overall survival.

### Example 8

This example describes analysis of the correlation between SPARC microenvironment signature and plasma SPARC levels.

As described in Examples 5 and 7 above, plasma SPARC levels and SMS was analyzed and the results combined to determine correlations for patient outcomes.

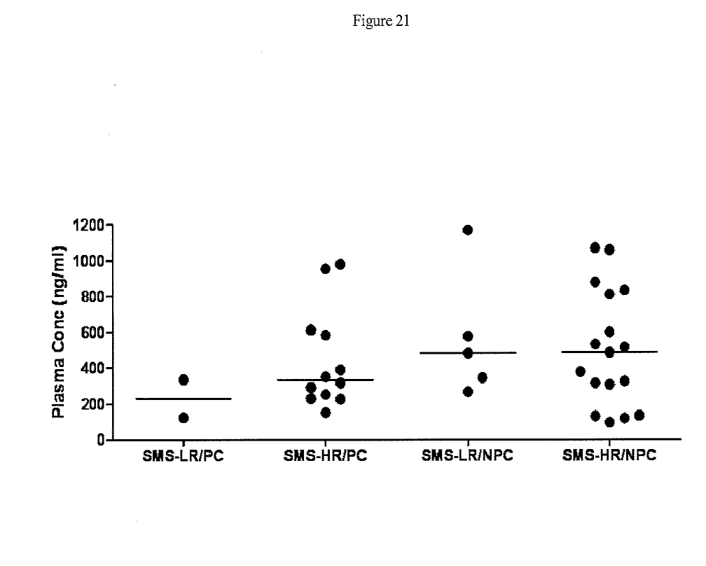

As shown in FIG. 21, baseline plasma SPARC was similar between SMS high-risk and SMS low-risk groups. Patients were coded as having a risk level of 0, 1, or 2, based on baseline plasma SPARC and SMS high risk versus low risk. A risk level of 0 is identified as low baseline plasma SPARC, with SMS low risk. A risk level of 1 is identified as high baseline plasma SPARC or SMS high risk. A risk level of 2 is identified as high baseline plasma SPARC and SMS high risk. Data for overall survival and progression free survival are shown in Table 4.

TABLE 4

| | Median | % Progression | Median | |
|---|---|---|---|---|
| | Progression Free | Free | Overall | % Overall |
| | Survival | Survival at | Survival | Survival at 12 |
| | (months) | 6 months | (months) | months |
| 0 Risk | 6.1 | 50% | 14.1 | 50% |
| 1 Risk | 4.1 | 21% | 14.4 | 53% |
| 2 Risks | 3.6 | 25% | 9.5 | 33% |

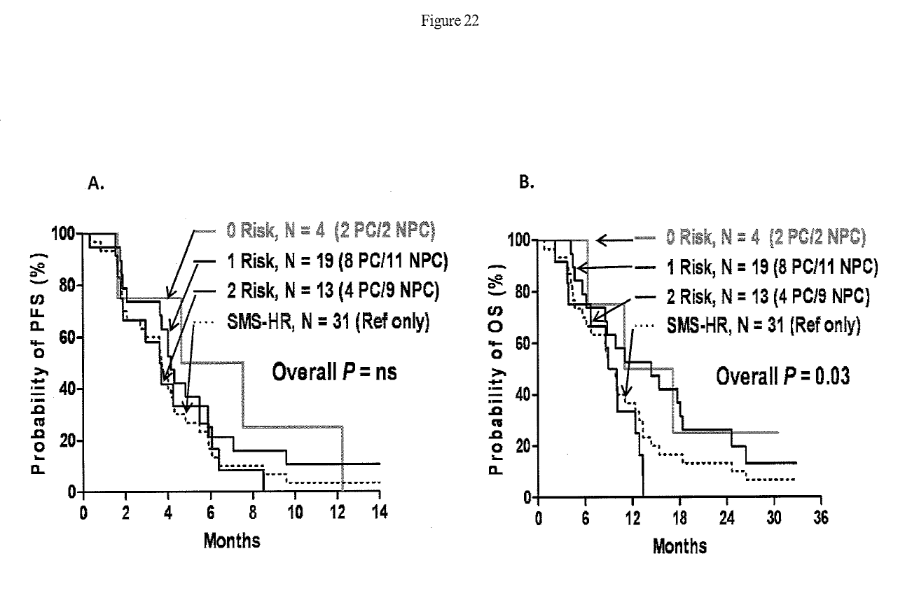

As shown in FIG. 22A-B, there was a general trend to worse progression free survival and overall survival with increasing risk level, although results were not significantly different for progression free survival of patients in the 2 Risks group.

These results show that patients with high plasma SPARC and high-risk SMS had significantly worse overall survival.

### Example 9

This example describes tumor xenograft assays demonstrating that SPARC negates the effectiveness of certain chemotherapies.

Female and male athymic NCr-nu mice between 5 and 6 weeks of age weighing approximately 20 g were purchased from Harlan, Inc. (Madison, Wis., USA). Human cancer cells HT29 (colon), PC3 (prostate), and MDA-MB-231 (breast) were propagated in cell culture and implanted subcutaneously at one million cells per flank of female (for MDA-MB-231 and HT29) or male (for PC3 prostate tumor) nude mice and allowed to grow to approximately 60-100 mm³ before treatment was initiated. Treatments included 5-fluorouracil (5FU), docetaxel (Taxotere®), albumin-bound paclitaxel (nab-paclitaxel), nab-paclitaxel plus suntinib malate (Sutent®), or nab-paclitaxel plus bevacizumab (Avastin®) with or without exogenously administered SPARC). Control animals for each xenograft were administered PBS. The longest (length) and shortest (width) tumor diameters (millimeter) and tumor depth were measured twice weekly. The tumor volume was calculated with the formula: tumor volume (mm3)=width×length×depth. Tumor growth inhibition (TGI) was defined as the percentage of tumor volume reduction compared with the control group at the time of euthanasia for the control animals. Tumor doubling time was defined as the time required for the tumor volume to double twice. Animal weights were measured twice weekly. Statistical analysis was performed using the Prism program (GraphPad, San Diego, Calif., USA). Analysis of variance (ANOVA) statistic was used to compare tumor growth curves.

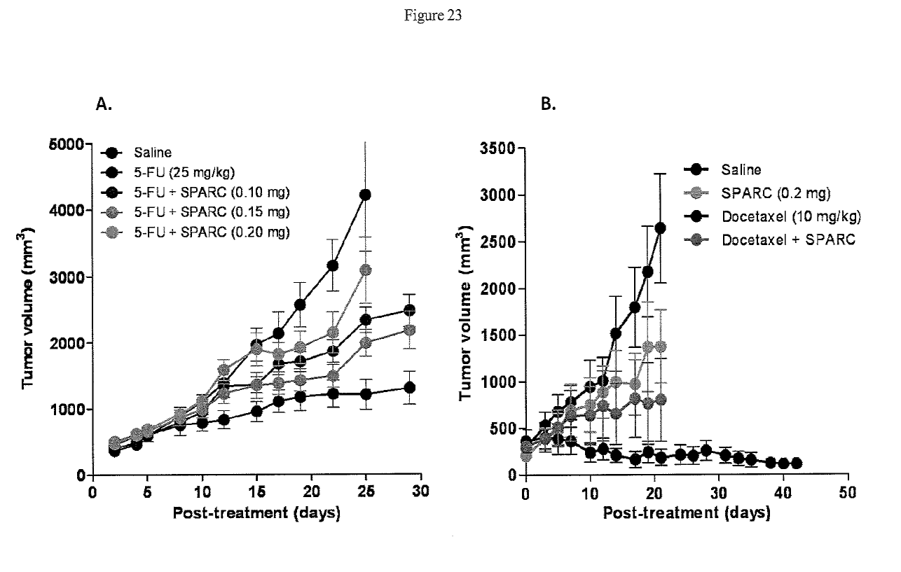

The impact of SPARC administration was evaluated on treatment with 5-fluorouracil. In the HT29 colon cancer xenograft model, 5-FU was effective in suppressing tumor growth (TGI 89.8%, P<0.0001 vs saline) without decrease in body weight. As shown in FIG. 23A, administration of SPARC caused dose dependent inhibition of 5-FU antitumor activity resulting in TGI of 50.8%, 47.4%, and 10.4%, respectively at 4, 6, and 8 mg/kg dose levels (P=0.003 vs 5-FU arm, Wilcoxon rank sum test). SPARC alone had modest antitumor activity (35.4%; NS). Similar results were obtained for docetaxel (FIG. 23B).

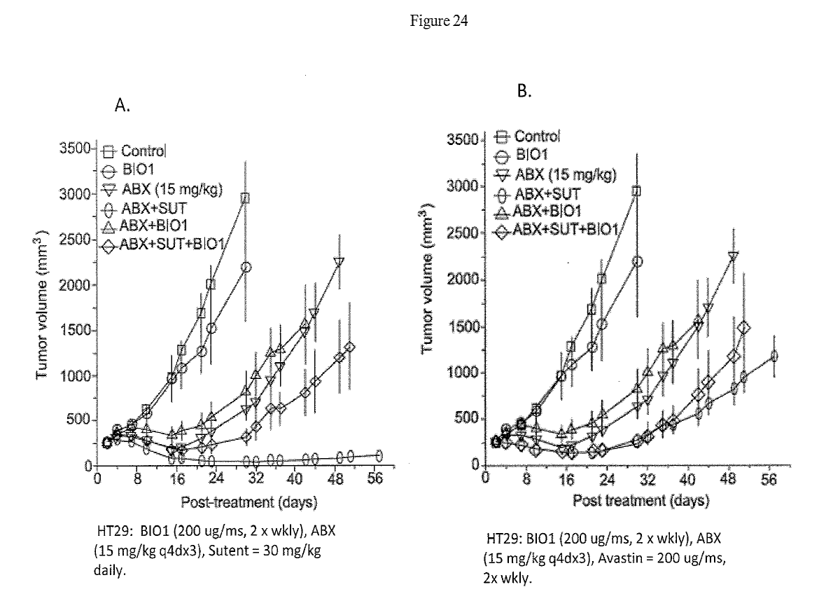

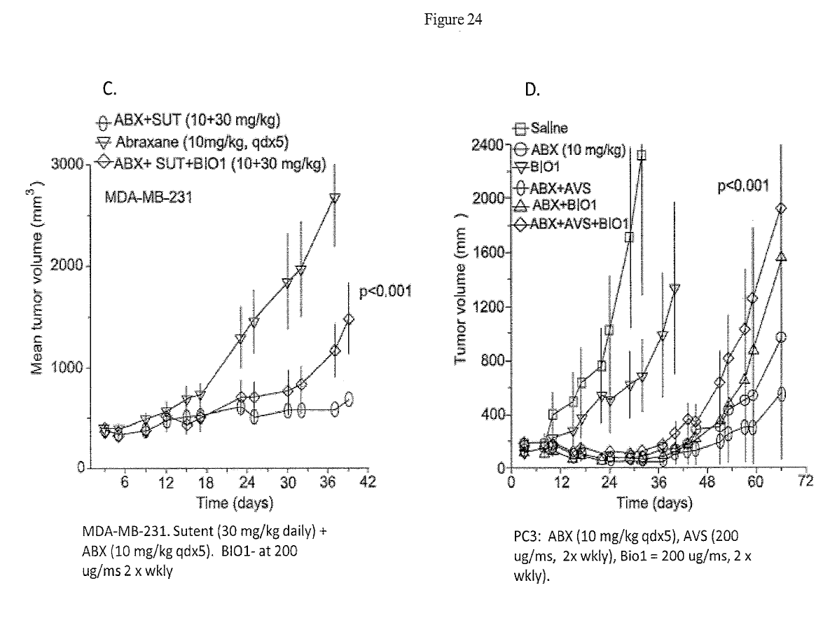

The impact of SPARC administration was also evaluated in the same HT29 xenograft model in combination with nab-paclitaxel at 15 mg/kg, every four days, for three cycles. Combination with antiangiogenic agent suntinib (Sutent®) significantly enhanced nab-paclitaxel (TGI >100% vs. 94.8%, P=0.015 vs nab-paclitaxel monotherapy, Wilcoxon rank sum test). In contrast, administration of SPARC significantly reduced nab-paclitaxel antitumor activity (TGI 84.8% vs. 94.8%, P=0.007 vs. nab-paclitaxel monotherapy, Wilcoxon rank sum test). More importantly, exogenous SPARC largely abolished the synergy of nab-paclitaxel with suntinib (TGI of 52.3% vs >100% (post day-51), P=0.006 vs. nab-paclitaxel plus suntinib combination arm, Wilcoxon rank sum test) (FIG. 24A). Treatment with nab-paclitaxel plus suntinib and/or SPARC was well-tolerated with no apparent weight loss. Similarly, bevacizumab alone induced significant TGI (75%, P<0.001). However, though the antitumor activity of nab-paclitaxel was enhanced by bevacizumab in this experiment, treatment with SPARC resulted only in minimal negative impact on the nab-paclitaxel/bevacizumab combination (FIG. 24B). Treatment with nab-paclitaxel plus bevacizumab and/or SPARC was well-tolerated with no apparent weight loss.

These findings were further confirmed in the MDA-MB-231 breast cancer xenografts (FIG. 24C) and PC3 prostate cancer xenografts (FIG. 24D). In these assays, the antitumor effects observed with the combinations of nab-paclitaxel and suntinib or nab-paclitaxel and bevacizumab were significantly inhibited by administration of SPARC (P<0.001, FIGS. 24C and 24D). In these experiments, nab-paclitaxel was dosed sub-optimally at 10 mg/kg, daily for five cycles.

These data show that exogenous SPARC promotes tumor growth and negates therapeutic benefits of chemotherapies such as nab-paclitaxel, docetaxel, and 5-fluorouracil.

### Example 10

In this example, the angiogenic behavior of SPARC was studied it two in vitro assays.

First, the effect of SPARC on sprouting was studied in an in vitro angiogenesis model system that was prepared using human umbilical cord vein endothelial cells (HUVEC) as described by Nakatsu et al (Methods Enzymol. 443:65-82 (2008)). Low passage HUVEC grown in M199 media supplemented with 10% FBS (Gibco, Carlsbad, Calif.) were switched to EGM-2 media (Clonetics, Walkersville, Md.) 2 days before beading. Cytodex 3 microcarrier beads (Amersham Pharmacia Biotech, Piscataway, N.J.) were hydrated and washed with PBS (pH 7.4). 1×10⁶ HUVEC cells were incubated with 2500 hydrated and sterilized beads in EGM-2 medium for 4 hours at 37° C. for coating. The coated beads were next embedded in 2 mg/mL fibrinogen in PBS with 0.15 units/mL aprotinin at a concentration of 500 beads/mL. Next, 0.625 units/mL thrombin was added and 0.5 mL of the mixture was added to each well of a 24 well plate. The solution was allowed to clot at RT for 5 minutes and 37° C. for another 15 min. After clotting 20,000 lung fibroblast cells in EGM-2 media were seeded in each well. Either 1, 10 or 100 μg/mL BioI SPARC protein in PBS, or PBS without protein was added to the growth medium and cultures were maintained for 5 days until sprouting was evaluated by light microscopy. The number of tubes formed/bead and the morphology of sprouting HUVEC cells in the cultures were determined using image pro software (Media Cybernetics, Bethesda, Md.).

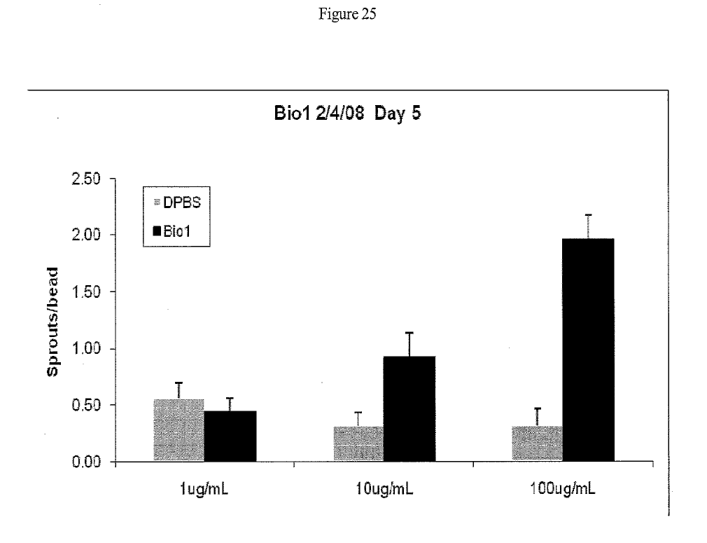

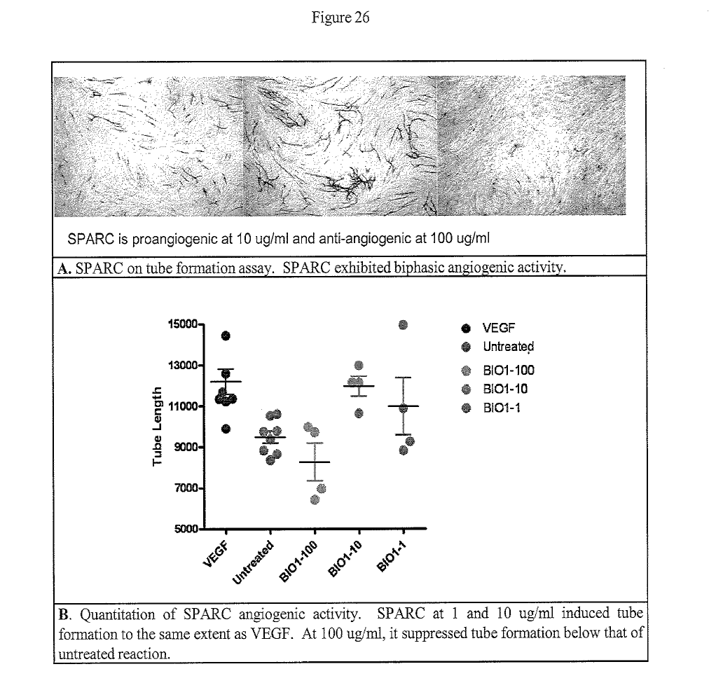

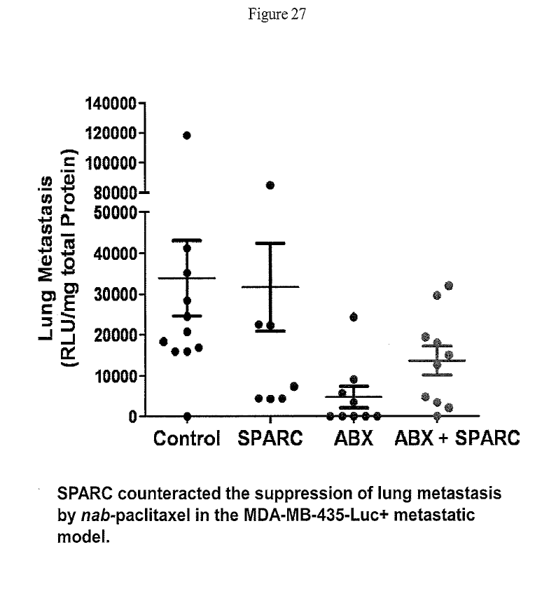

The results of this assay show that treatment with SPARC induced sprouting in a dose dependent manner (FIG. 25). At 1 μg/mL less than 50 percent of the seeded HUVEC/beads had developed sprouts. Addition of 10 or 100 μg/mL SPARC resulted in an average of 0.9 or 1.9 sprouts/bead respectively (FIG. 26B) whereas the average sprout number in cultures without SPARC remained below about 0.5 sprouts/bead.

Next, the effect of SPARC on tubule formation was examined using the TCS cellworks human Angiokit model kit (TCS-ZHA-1000, TCS Cell Works Buckingham, UK). In this assay, the endothelial cells initially form small islands within the culture matrix. They subsequently begin to proliferate and then enter a migratory phase during which they move through the matrix to form threadlike tubule structures. These gradually join up (by 9-11 days) to form a network of anastomosing tubules which closely resembles a capillary bed.

24-well pre-seeded endothelial cell tissue culture plates containing early stage co-cultures (day 2-3) were used according to manufacturer's recommendations (TCS Cell Works, Buckingham, UK). The cultures were incubated at 37° C./5% CO₂ for 11 days. Media on the cultures was changed with added test and control compounds on days 4, 7 and 9 after initial treatment, followed by fixation with 70% ethanol on day 11. Fixed cultures were subsequently stained as follows: Primary rabbit anti-CD31 antibody (Thermo Scientific) was diluted to a final concentration of 2 μg/mL in PBS/1% BSA blocking buffer. Then, 0.5 mL of diluted anti-CD31 antibody was added to each well and incubated for 1 h at 37° C. The cultures were washed three times with PBS before AP conjugated secondary antibody (Thermo Scientific, 1 μg/mL) was added and incubated for 1 hour at 37° C. followed by extensive washing with PBS and water. For visualization of formed tubules, 0.5 mL of 1 step NIB/BCIP solution (Thermo Scientific) was added per well and incubated until staining was complete. The reaction was then stopped with water.

The number and lengths of tubules formed were then evaluated by light microscopy. Comparison of tubule development was conducted using the “AngioSys” image analysis system developed specifically for the analysis of images produced using the AngioKit (TCS Cell Works, Buckingham, UK). Stained tubules captured by the analysis software are reduced in width to a single pixel. The total number of pixels in a given field of view therefore represents the length of tubules. From this data, mean tubule length, standard deviations and coefficients of variation can be calculated. Total vessel number, total tubule area and the number of branch points can be similarly determined.

As shown in FIG. 26, in this system, SPARC exhibited biphasic angiogenic activity. These results show that low concentrations of SPARC (1 and 10 μg/mL) can stimulate angiogenesis, particularly concentrations of 10 μg/mL, while high concentration of SPARC (100 μg/mL) significantly inhibited angiogenesis.

The results of these two assays show that, at least in certain concentrations, the activity of circulating SPARC stimulates angiogenesis.

### Example 11

This example illustrates the effects of exogenous SPARC and nab-paclitaxel on tumor progression in the MDA-435 metastatic model.

Female and male athymic NCr-nu mice between 5 and 6 weeks of age weighing approximately 20 g were purchased from Harlan, Inc. (Madison, Wis., USA). MDA-MB-435-Luc+ were implanted orthotopic in mammary fat pad (MFP) at 4×10⁶ cells in 50% Matrigel and allowed to reach average tumor volume of 180 mm³ before treatment. Treatments were: 2 cycles of 10 mg/kg nab-paclitaxel, 4 mg/kg SPARC, and bevacizumab biweekly. At appropriate time interval, the following organs were harvested for quantitation of metastasis: Proximal lymph nodes, contralateral lymph nodes, lungs, liver and brain. Statistical tests used included t-test and Mann Whitney-U.

Mice were monitored 2-3 times a week and tumor growth was recorded. Mice were sorted when tumor volumes reached 180 mm³, and the first cycle of treatment was administered. Seven rest days were given between the first and second cycle. Bevacizumab and SPARC were continually administered during the rest period. Weight was measured periodically throughout the study to assess toxic effects from the therapy. Nnab-paclitaxel caused minimal weight loss which was rapidly regained after the drug was discontinued (data not presented).

The following dosing schedules were followed: Control (Group 1); 10 mg/kg nab-paclitaxel daily for 3 to 5 cycles (Group 2); 4 mg/kg soluble SPARC (Bio 1) injected i.v. biweekly (Group 3); and 10 mg/kg nab-paclitaxel daily for two to five cycles and 4 mg/kg SPARC-biweekly (Group 4).

Control tumors had steady tumor growth with an average increase in volume of 43.75±5.65 mm3/day and a final volume of 1870.6 mm3 in 56 days. SPARC treated group exhibited a similar growth rate of 23.16±4.38 mm3/day and an average volume of 1765 mm3 in 63 days. The nab-paclitaxel alone group had 18 days tumor regression, which resulted in 76% reduction in tumor volume before regrowth occurred. Five out of 9 mice had palpable tumors; however all have grown to a final average volume of 739 mm3 at a rate of 22.98±0.89 mm3/day. The regrowth of the tumors occurred 6 days after the cessation of nab-paclitaxel treatment. The regression lasted for 29 days and regrowth occurred at 6.10±1.16 mm3/day to a final volume of 136 mm3. The nab-paclitaxel+SPARC group behaved similarly to the nab-paclitaxel group. Two complete regressions occurred, but regrowth occurred 21 days after treatment was started at a rate of 21.05±2.57 mm3/day to a final volume of 653 mm3. (Table 4).

TABLE 4

| | Tumor | | p-value in | |
|---|---|---|---|---|
| | Volume | | relation to | p-value in |
| Group | on day 56 | % Inhibition | control | relation to ABX |
| Group 1 - | 1775 | — | — | — |
| Control | | | | |
| Group 2 - nab- | 542 | 76 | <0.0001 | — |
| paclitaxel | | | | |
| Group 3 - | 1524 | 35 | ns | — |
| SPARC | | | | |
| Group 4 - nab- | 487 | 82 | <0.0001 | ns |
| paclitaxel + | | | | |
| SPARC | | | | |

Total metastatic burden was also calculated. It is presented as measured luciferase activity in tissue extracts expressed in RLU normalized per mg protein. All contralateral lymph nodes were negative for all groups. There were a few incidences of metastasis occurred in the proximal lymph node, liver and brain. The proximal lymph showed metastasis in 2 out of 12 animals in the control group. Liver metastasis occurred somewhat frequently in the both control and SPARC groups (present in 3 mice in each group). There was also one control mouse that had brain metastasis.

The majority of metastasis occurred in the lungs. The control group had 11 of 12 mice positive with an average RLU/mg total protein of 33840±9176 (N=12). SPARC had very little effect on metastasis with 9 of 9 mice positive with an average RLU/mg total protein of 31630±10820 (N=9). Nab-paclitaxel alone had some effect on lung metastasis with only 4 out 9 mice positive and RLU/mg total protein of 4722+2684 (N=9), p=0.015 versus control (Student's t-test). SPARC co-administered with nab-paclitaxel increased the incidence to 9 of 10 mice positive and RLU/mg total protein of 13690±3579 (N=10), p=ns versus control (Student's t-test). These data demonstrate that circulating SPARC negates the anti-metastatic activity of nab-paclitaxel.

Because circulating platelet and macrophages express high levels of SPARC and could be the source of plasma SPARC (Sangaletti S. et al., Cancer Res. 68: 9050-9059 (2008); Sangaletti S. et al., J. Exp. Med. 198: 1475-1485 (2003)), these results show that under certain circumstances, elevated SPARC either experimentally (i.e., by exogenous administered SPARC) or by inherent overexpression of circulating SPARC (i.e., by SPARC expressing organs in the patients such as tumor, leukocytes, platelets, and macrophages), SPARC increases metastatic risk in the presence of chemotherapy.

### Example 12

This example describes the use of a Tube Formation Assay to determine the effect of monoclonal SPARC binding antibodies Imm12, Imm14, and mHTI on the angiogenic behavior of SPARC.

This assay was performed using the TCS Cellworks human AngioKit model kit (TCS-ZHA-1000, TCS Cell Works, Buckingham, UK) as described in Example 10 above. The media contained 10 μg/mL recombinant human SPARC protein. The media also contained 300 μg/mL of one of the above prepared mouse SPARC binding monoclonal antibodies (Imm12, Imm14, mHTI), or a control (mouse IgG).

To examine the effect of SPARC on tubule formation, and interference with that function by SPARC binding monoclonal antibodies, the number of tubules formed and their length was evaluated by light microscopy.

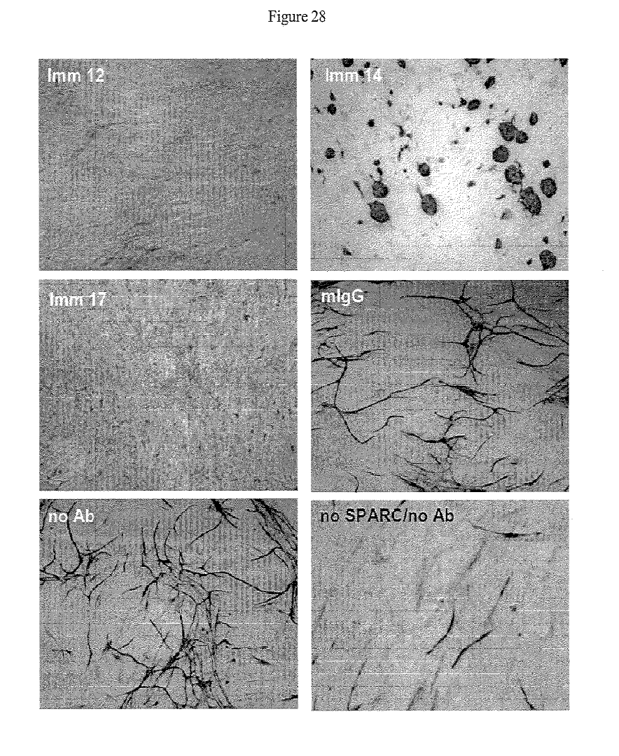

The results of this assay are presented in FIG. 28. The presence of SPARC in the culture media resulted in the formation of numerous long tubules. These results confirmed the pro-angiogenic effect of SPARC in this model system. However, addition of any of the 3 anti-SPARC antibodies resulted in inhibition of this effect. In presence of either Imm12 or Imm14 monoclonal antibody only very slight formation of very short tubules was observed. Addition of mHTI to the culture media lead to an almost complete inhibition of the tube formation.

These results show that SPARC binding antibodies Imm12, Imm14, and mHTI can overcome the angiogenesis-stimulating effect of SPARC.

### Example 13

This example demonstrates that mHTI, Imm12, and Imm14 do not localize at a tumor site in an in vivo animal model.

Nude mice implanted with subcutaneous HT29 colon xenografts were treated with Imm series antibodies labeled with labeled with Alexa 680 fluorescent dye at dose of 200 ug/mouse. The labeled Imm antibodies were formulated in saline and administered intravenously on day 1. The fluorescent signal was followed in these mice over the course of 36 days.

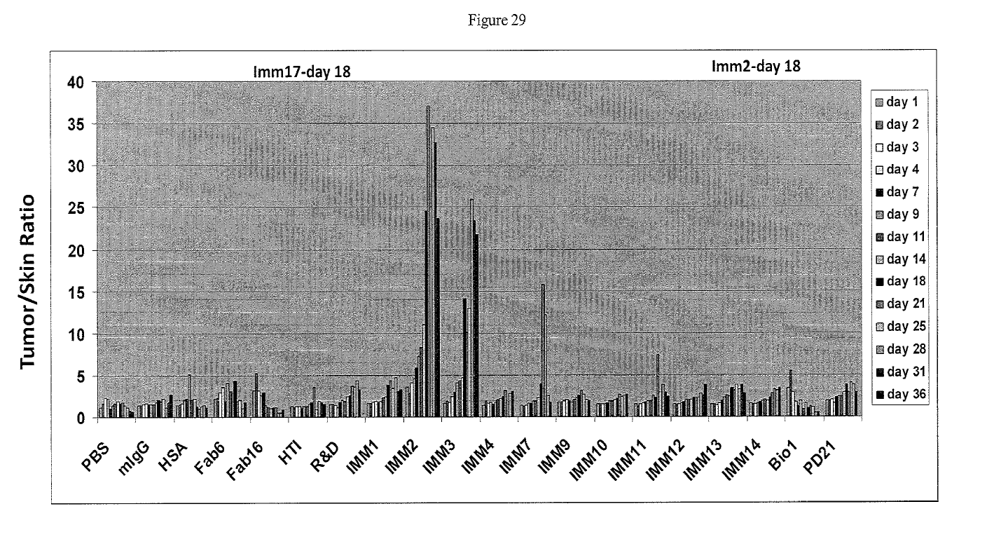

The results of this study, which are presented in FIG. 29, indicate that mHTI (labeled “HTI”), Imm12 and Imm14 do not localize at the tumor site in this model, while Imm2 localizes well.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A composition comprising a SPARC binding antibody, wherein the SPARC binding antibody comprises Imm12, Imm14, hHTI, or a combination thereof.

2. The composition of claim 1 wherein the SPARC binding antibody comprises hHTI.

3. The composition of claim 1 wherein the SPARC binding antibody comprises Imm12.

4. The composition of claim 1 wherein the SPARC binding antibody comprises Imm14.

5. The composition of claim 1, further comprising an active agent, wherein the active agent is conjugated to the SPARC binding antibody.

6. The composition of claim 5, wherein the active agent comprises a therapeutic agent or a diagnostic agent.

7. The composition of claim 6, wherein the therapeutic agent or diagnostic agent is a therapeutic agent selected from the group consisting of tyrosine kinase inhibitors, kinase inhibitors, biologically active agents, biological molecules, radionuclides, adriamycin, ansamycin antibiotics, asparaginase, bleomycin, busulphan, cisplatin, carboplatin, carmustine, capecitabine, chlorambucil, cytarabine, cyclophosphamide, camptothecin, dacarbazine, dactinomycin, daunorubicin, dexrazoxane, docetaxel, doxorubicin, etoposide, epothilones, floxuridine, fludarabine, fluorouracil, gemcitabine, hydroxyurea, idarubicin, ifosfamide, irinotecan, lomustine, mechlorethamine, mercaptopurine, meplhalan, methotrexate, rapamycin (sirolimus), mitomycin, mitotane, mitoxantrone, nitrosurea, paclitaxel, pamidronate, pentostatin, plicamycin, procarbazine, rituximab, streptozocin, teniposide, thioguanine, thiotepa, taxanes, vinblastine, vincristine, vinorelbine, taxol, combretastatins, discodermolides, transplatinum, anti-vascular endothelial growth factor compounds (“anti-VEGFs”), anti-epidermal growth factor receptor compounds (“anti-EGFRs”), 5-fluorouracil and derivatives, radionuclides, polypeptide toxins, apoptosis inducers, therapy sensitizers, enzyme or active fragment thereof, and combinations thereof.

8. The composition of claim 6, wherein the therapeutic agent or diagnostic agent is a therapeutic agent comprising an antibody or antibody fragment.

9. The composition of claim 8, wherein said antibody or antibody fragment is a Fc fragment of IgG, or IgA, or IgD, or IgE, or IgM.

10. The composition of claim 8, wherein said antibody or antibody fragment mediates one or more of complement activation, cell mediated cytotoxicity or opsonization, or mast cell activation, or other immune response.

11. The composition of claim 6, wherein the therapeutic agent or diagnostic agent is a diagnostic agent selected from the group consisting of fluorochromes, radioactive agents, MRI contrast agents, X-ray contrast agents, ultrasound contrast agents, and PET contrast agents.

12. The composition of claim 1, wherein the composition is contained in a liposome.

13. The composition of claim 1, wherein the composition is contained in an albumin nanoparticle.

14. The composition of claim 1, wherein the composition further comprises a suitable pharmaceutical carrier.

15. The composition of claim 1, wherein said composition is administered to a patient via i.v., topically, via injection, via inhalation, intranasally, rectally or orally.

16. A method for diagnosing or treating a disease in an animal comprising administering a diagnostically or therapeutically effective amount of a composition comprising a SPARC binding antibody, wherein the SPARC binding antibody comprises Imm12, Imm14, hHTI, or a combination thereof.

17. The method of claim 16 wherein the SPARC binding antibody comprises hHTI.

18. The method of claim 16 wherein the SPARC binding antibody comprises Imm12.

19. The method of claim 16 wherein the SPARC binding antibody comprises Imm14.

20. The method of claim 16, wherein the composition further comprises an active agent conjugated to the SPARC binding antibody

21. The method of claim 20, wherein the active agent comprises a therapeutic agent or a diagnostic agent.

22. The method of claim 21, wherein the therapeutic agent or diagnostic agent is a therapeutic agent selected from the group consisting of tyrosine kinase inhibitors, kinase inhibitors, biologically active agents, biological molecules, radionuclides, adriamycin, ansamycin antibiotics, asparaginase, bleomycin, busulphan, cisplatin, carboplatin, carmustine, capecitabine, chlorambucil, cytarabine, cyclophosphamide, camptothecin, dacarbazine, dactinomycin, daunorubicin, dexrazoxane, docetaxel, doxorubicin, etoposide, epothilones, floxuridine, fludarabine, fluorouracil, gemcitabine, hydroxyurea, idarubicin, ifosfamide, irinotecan, lomustine, mechlorethamine, mercaptopurine, meplhalan, methotrexate, rapamycin (sirolimus), mitomycin, mitotane, mitoxantrone, nitrosurea, paclitaxel, pamidronate, pentostatin, plicamycin, procarbazine, rituximab, streptozocin, teniposide, thioguanine, thiotepa, taxanes, vinblastine, vincristine, vinorelbine, taxol, combretastatins, discodermolides, transplatinum, anti-vascular endothelial growth factor compounds (“anti-VEGFs”), anti-epidermal growth factor receptor compounds (“anti-EGFRs”), 5-fluorouracil and derivatives, radionuclides, polypeptide toxins, apoptosis inducers, therapy sensitizers, and enzymes or active fragment thereof.

23. The method of claim 21, wherein the therapeutic agent or diagnostic agent is a therapeutic agent comprising an antibody or antibody fragment.

24. The method of claim 23, wherein the antibody or antibody fragment is a Fc fragment of IgG, or IgA, or IgD, or IgE, or IgM.

25. The method of claim 23, wherein the antibody or antibody fragment mediates one or more of complement activation, cell mediated cytotoxicity or opsonization, or mast cell activation, or other immune response.

26. The method of claim 21, wherein the therapeutic agent or diagnostic agent is a diagnostic agent selected from the group consisting of fluorchromes, radioactive agents, MRI contrast agents, X-ray contrast agents, ultrasound contrast agents, and PET contrast agents.

27. The method of claim 16, wherein the composition further comprises a suitable pharmaceutical carrier.

28. The method of claim 16, wherein the therapeutically effective amount of the composition is administered to a patient via i.v., topically, via injection, via inhalation, intranasally, rectally or orally.

29. The method of claim 16, further comprising administering a therapeutically effect amount of albumin bound nanoparticulate paclitaxel.

30. The method of claim 16, wherein the tumor is selected from the group consisting of oral cavity tumors, pharyngeal tumors, digestive system tumors, respiratory system tumors, bone tumors, cartilaginous tumors, bone metastases, sarcomas, skin tumors, melanoma, breast tumors, genital system tumors, urinary tract tumors, orbital tumors, brain and central nervous system tumors, gliomas, endocrine system tumors, thyroid tumors, esophageal tumors, gastric tumors, small intestinal tumors, colonic tumors, rectal tumors, anal tumors, liver tumors, gall bladder tumors, pancreatic tumors, laryngeal tumors, tumors of the lung, bronchial tumors, non-small cell lung carcinoma, small cell lung carcinoma, uterine cervical tumors, uterine corpus tumors, ovarian tumors, vulvar tumors, vaginal tumors, prostate tumors, prostatic carcinoma, testicular tumors, tumors of the penis, urinary bladder tumors, tumors of the kidney, tumors of the renal pelvis, tumors of the ureter, head and neck tumors, parathyroid cancer, Hodgkin's disease, Non-Hodgkin's lymphoma, multiple myeloma, leukemia, acute lymphocytic leukemia, chronic lymphocytic leukemia, acute myeloid leukemia, chronic myeloid leukemia.

31. The method of claim 16, wherein the animal is human.

32. A method of treating a tumor in an animal with an anticancer agent and a SPARC binding antibody comprising:
(a) isolating a biological sample from the animal;
(b) detecting the expression of SPARC protein in the biological sample; and
(c) quantifying the amount of SPARC protein in the biological sample;
wherein if the SPARC protein is present above a threshold level, administering a therapeutically effective amount of the anticancer agent and a therapeutically effective amount of the SPARC binding antibody; or
if the SPARC protein is present below the threshold level, administering a therapeutically effective amount of the anticancer agent and none of the SPARC binding antibody.

33. The method of claim 32, wherein the SPARC binding antibody comprises Imm12, Imm14, hHTI, or a combination thereof.

34. The method of claim 32, wherein the biological sample is isolated from a bodily fluid.

35. The method of claim 34, wherein the bodily fluid is selected from the group consisting of cerebrospinal fluid, blood, plasma, serum, and urine.

36. The method of claim 32, wherein the animal is a human.

37. The method of claim 32, wherein the tumor is selected from the group consisting of oral cavity tumors, pharyngeal tumors, digestive system tumors, respiratory system tumors, bone tumors, cartilaginous tumors, bone metastases, sarcomas, skin tumors, melanoma, breast tumors, genital system tumors, urinary tract tumors, orbital tumors, brain and central nervous system tumors, gliomas, endocrine system tumors, thyroid tumors, esophageal tumors, gastric tumors, small intestinal tumors, colonic tumors, rectal tumors, anal tumors, liver tumors, gall bladder tumors, pancreatic tumors, laryngeal tumors, tumors of the lung, bronchial tumors, non-small cell lung carcinoma, small cell lung carcinoma, uterine cervical tumors, uterine corpus tumors, ovarian tumors, vulvar tumors, vaginal tumors, prostate tumors, prostatic carcinoma, testicular tumors, tumors of the penis, urinary bladder tumors, tumors of the kidney, tumors of the renal pelvis, tumors of the ureter, head and neck tumors, parathyroid cancer, Hodgkin's disease, Non-Hodgkin's lymphoma, multiple myeloma, leukemia, acute lymphocytic leukemia, chronic lymphocytic leukemia, acute myeloid leukemia, chronic myeloid leukemia.

38. The method of claim 32, wherein the chemotherapeutic or anticancer agent is selected from the group consisting of docetaxel, paclitaxel, taxanes, platinum compounds, antifolates, antimetabolites, antimitotics, DNA damaging agents, proapoptotics, differentiation inducing agents, antiangiogenic agents, antibiotics, hormones, peptides, antibodies, and combinations thereof.

39. The method of claim 32, wherein treating the animal further comprises administering a therapeutically effective amount of an angiogenesis inhibitor.

40. The method of claim 39, wherein the angiogenesis inhibitor is selected from the group consisting of bevacizumab, suntinib, HKP, IFN-alpha, fumagillin, angiostatin, endostatin, thalidomide, and combinations thereof.

41. The method of claim 32, wherein the chemotherapeutic agent comprises particles of protein-bound drug.

42. The method of claim 40, wherein the protein component of the protein-bound drug particles comprises albumin.

43. The method of claim 41, wherein the chemotherapeutic agent comprises particles of albumin-bound paclitaxel.

44. The method of claim 32, wherein the expression of SPARC protein is detected with an antibody.

45. The method of claim 32, wherein the expression of SPARC protein is detected without an antibody.

46. The method of claim 32, wherein the expression of SPARC protein is detected with a non-antibody SPARC binding molecule.

47. The method of claim 32, wherein the expression of SPARC protein is detected without using a SPARC binding molecule.
